(19) United States
(12) Patent Application Publication
(10) Pub. No.: US 2022/0118209 A1
(43) Pub. Date: Apr. 21, 2022

(54) PRESSURE RELIEF DEVICE AND COMPONENTS THEREFOR

(71) Applicant: Fisher & Paykel Healthcare Limited, Auckland (NZ)

(72) Inventors: Taylor James EDWARDS, Auckland (NZ); Arjen David KAT, Auckland (NZ); Shane Terry MASSEY, Auckland (NZ); Hamish Adrian OSBORNE, Auckland (NZ); Cameron Leslie MATTHEWS, Auckland (NZ); Rhys John EVANS, Auckland (NZ); Evan Anthony MAUNDER, Auckland (NZ); Beth Frances HARDY, Auckland (NZ); Aidan Robert BURGESS, Auckland (NZ); Leo Mckenzie RODGER, Auckland (NZ)

(21) Appl. No.: 17/425,269

(22) PCT Filed: Jan. 31, 2020
(86) PCT No.: PCT/IB2020/050766

§ 371 Date: Jul. 22, 2021

Related U.S. Application Data
(60) Provisional application No. 62/913,896, filed on Oct. 11, 2019.
(60) Provisional application No. 62/799,952, filed on Feb. 1, 2019.

(51) Int. Cl. A61M 16/08; A61M 16/06; A61M 16/01; A61M 16/20; A61M 16/10

(52) U.S. Cl. CPC A61M 16/0858; A61M 16/0672; A61M 16/01; A61M 2016/0027; A61M 16/0875; A61M 16/0866; A61M 16/107; A61M 16/209

(57) ABSTRACT

A connector having a connector body with an inlet and an outlet defining a gas flow passage therebetween. The connector body has an overlap portion that is configured to overlap with a portion of a second connector when connected. An access passage extends through the overlap portion to the gas flow passage.

### TECHNICAL FIELD

The present disclosure relates to a pressure relief device for medical systems for conveying gases to and/or from a patient, in particular a flow and/or pressure compensating pressure relief device, a diaphragm component, and a connector therefor.

### BACKGROUND

Respiratory gas supply systems provide gas for delivery to a patient. Respiratory gas supply systems typically include a fluid connection between a gas supply and the patient. This may include an inspiratory tube and a patient interface. Such systems include a number of different components to ensure gas is correctly delivered to a patient. Many of the components are single use components that are disposed of after each use, while other components are multi-use components. In some situations, multi-use components are preferred. In some situations, it is necessary to connect single use components to multi-use components. However, this can cause problems if single use components are incorrectly or inadvertently assembled with multi-use components. In addition, some components are complex products that have a number of different features and functions. The design and/or production of such components cannot be readily altered or modified.

In pressure relief valves containing flexible diaphragms, the diaphragm(s) can be susceptible to oscillations during normal use, due to resonance of the diaphragm and fluctuations in pressure in the chamber adjacent to the diaphragm. These oscillations cause noise and decrease the stability of the valve, particularly when the diaphragm lifts from the valve seat. Larger and higher frequency oscillations are associated with lower stability and higher noise levels. Such oscillations may also increase hysteresis in the valve, that is, increase the lag time for flow to be restored after a blockage of the conduit has been removed.

### SUMMARY

It is therefore an object of certain embodiments disclosed herein to provide a connector that will go at least some way towards addressing the foregoing problems or which will at least provide the industry with a useful choice.

In a first aspect, there is provided a connector comprising: a connector body having an inlet and an outlet defining a gas flow passage there between; the connector body having an overlap portion that is configured to overlap with a portion of a second connector when connected; and an access passage extending through the overlap portion to the gas flow passage.

The access passage may comprise an aperture to fluidly communicate with the gas flow passage to sense pressure in the gas flow passage.

The gas flow passage may be defined at least in part by a wall, and the access passage may comprises an aperture in the wall of the connector.

The connector may further comprise a cavity forming portion configured to form a cavity with the second connector.

The cavity forming portion may comprise an arcuate surface.

The cavity forming portion may be a recess in a surface of the connector body.

The cavity forming portion may be in fluid communication with the gas flow passage via the access passage.

The cavity forming portion may have a longitudinal dimension that may be substantially parallel to a direction of gas flow in the gas flow passage.

The connector may further comprise a first sealing mechanism configured to form a first seal with a portion of the second connector.

The first sealing mechanism may comprise one or more of: a face seal, an O-ring, a lip seal, a wiper seal, or a sealing surface.

The overlap portion may comprise the first sealing mechanism.

The first sealing mechanism may comprise an internal or external sealing surface for friction/interference fit with the second connector.

The access and/or cavity forming portion may be arranged upstream of the first sealing mechanism.

The connector may further comprise a second sealing mechanism configured to form a second seal with a portion of the second connector.

The cavity forming portion may be between the first sealing mechanism and the second sealing mechanism.

The access passage may be positioned between the first sealing mechanism and the second sealing mechanism.

The second sealing mechanism may comprise one or more of: a face seal, an O-ring, a lip seal, a wiper seal, or a sealing surface.

The overlap portion may comprise the second sealing mechanism.

The second sealing mechanism may comprise an internal or external sealing surface for friction/interference fit with the second connector.

A portion and/or surface of the connector may be tapered.

The connector may further comprise one or more alignment features.

The aperture may be arranged substantially parallel or substantially perpendicular to a direction of gas flow in the gas flow passage.

The aperture may be radially arranged about the gas flow passage.

The connector may further comprise a stepped portion and the aperture may be arranged on the stepped portion.

The connector aperture may be in fluid communication with the gas flow passage via another aperture, the aperture may be in fluid communication by a channel.

The connector may further comprise a flow restriction.

The flow restriction may be provided at a terminal end of the connector.

The flow restriction may be arranged in a recess.

The flow restriction may be provided by a constriction spaced away from a terminal end of the connector.

The constriction may be a venturi.

The access passage may be provided at the flow restriction, or immediately adjacent and on a downstream side of the flow restriction.

The connector body may taper outwardly from the terminal end, from a smaller diameter to a larger diameter.

The connector may further comprise a stop.

The stop may be or may comprise a collar.

A surface of the collar may be configured to form a face seal with a surface of the second connector.

The connector may further comprise a radial clearance near the terminal end of the connector.

The cavity forming portion may be tapered relative to a direction of gas flow.

The gas flow passage may be or may comprise a pressure line.

The connector may taper towards a terminal end, from a smaller diameter to a larger diameter.

The connector may be configured to be coupled to a pressure relief valve.

The connector may further comprise an engagement mechanism configured to couple the connector to a pressure relief valve.

The connector may be integral with a pressure relief valve.

The pressure relief valve may be a flow and/or pressure compensated pressure relief valve.

The pressure line may be in fluid communication with a sensing chamber of the pressure relief valve.

The pressure relief valve may comprise a sensing member configured to sense a pressure differential between the sensing chamber and a main gas flow passage that provide gas flow to a patient.

Movement of the sensing member may change the venting pressure of a valve member.

The pressure line may be a first pressure line and the connector may further comprise a second pressure line that may be upstream of the first pressure line.

The connector may be configured to be coupled to a circuit component.

The connector may further comprise an engagement mechanism configured to engage the connector with the circuit component.

The first pressure line and the second pressure line may each be coupled to a pressure sensing mechanism.

In a second aspect, there is provided a connector comprising: an inlet and an outlet defining a gas flow passage therebetween; a flow restriction configured to restrict flow through the gas flow passage; and an access passage to the gas flow passage, the access passage being arranged downstream of the flow restriction.

The gas flow passage may be defined at least in part by a wall, and the access passage may comprise an aperture in the wall of the connector.

The flow restriction may be in a recess at the inlet.

A portion and/or surface of the connector may be tapered.

The access passage may be arranged between a first sealing mechanism and the flow restriction.

The access passage may be provided at the flow restriction, or immediately adjacent and on a downstream side of the flow restriction.

The first sealing mechanism may comprise one or more of: a face seal, an O-ring, a lip seal, a wiper seal, or a sealing surface.

The surface may comprise an arcuate surface.

The sealing surface may seal via a friction/interference fit with an internal surface of a second connector.

The connector may be a two-part connector, a first part comprising the flow restriction and a second part comprising the first sealing mechanism.

The first and second parts may be separated by a gap.

The first and second sections may be linked.

The flow restriction may be upstream of the first sealing mechanism.

The connector may further comprise a cavity forming portion configured to form a cavity with a second connector.

The connector cavity forming portion may comprise an external arcuate surface.

The cavity forming portion may be in fluid communication with the gas flow passage via the access passage.

The cavity forming portion may have a longitudinal dimension that may be substantially parallel to a direction of gas flow in the gas flow passage.

The connector may further comprise a second sealing mechanism arranged between the terminal end and the access passage and/or the cavity forming portion.

The second sealing mechanism may comprise one or more of: a face seal, an O-ring, a lip seal, a wiper seal, or a sealing surface.

The sealing surface may comprise an arcuate or curved surface.

The sealing surface may seal via a friction/interference fit with an internal surface of a second connector.

The connector, may further comprise a stop.

The stop may be or may comprise a collar.

A surface of the collar may be configured to form a face seal with a surface of the second connector.

The connector may be configured to connect to a second connector, the second connector having a pressure line that may be in fluid communication with the aperture.

In a third aspect, there is provided an assembly comprising: a first connector comprising a connector body having an inlet and an outlet defining a first connector gas flow passage therebetween, and an overlap portion; a second connector comprising a connector body having an inlet and an outlet defining a second connector gas flow passage therebetween, and an overlap portion; wherein the first connector and the second connector are configured to be assembled such that the overlap portion of the first connector and the overlap portion of the second connector are contiguous to form an overlapping connection and provide an assembly gas flow passage; and an access passage extending through the overlap connection to the assembly gas flow passage.

The access passage may comprise an aperture to fluidly communicate with the gas flow passage to sense pressure in the gas flow passage.

The gas flow passage may be defined at least in part by a wall, and the access passage may comprise an aperture in the wall of the connector.

The flow restriction may be in a recess at the inlet of the second connector.

A portion and/or surface of the connector may be tapered.

The access passage may be arranged between a first sealing mechanism and the flow restriction.

The access passage may be provided at the flow restriction, or immediately adjacent and on a downstream side of the flow restriction.

The first sealing mechanism may comprise one or more of: a face seal, an O-ring, a lip seal, a wiper seal, or a sealing surface.

The sealing surface may comprise an arcuate surface.

The sealing surface may seal via a friction/interference fit with an internal surface of a second connector.

The connector may be a two part connector, a first part comprising the flow restriction and a second part comprising the first sealing mechanism.

The first and second sections may be linked.

The flow restriction may be upstream of the first sealing mechanism.

The assembly may further comprise a cavity defined by the first connector and the second connector.

The cavity may be defined by an external arcuate surface of the first connector.

The cavity may be in fluid communication with the gas flow passage via the access passage.

The cavity may have a longitudinal dimension that may be substantially parallel to a direction of gas flow in the gas flow passage.

The assembly may further comprise a second sealing mechanism arranged between the terminal end and the access passage and/or the cavity.

The second sealing mechanism may comprise one or more of: a face seal, an O-ring, a lip seal, a wiper seal, or a sealing surface.

The sealing surface may comprise an arcuate or curved surface.

The sealing surface may seal via a friction/interference fit with an internal surface of a second connector.

The second connector further may comprise a stop.

The stop may be or may comprise a collar.

A surface of the collar may be configured to form a face seal with a surface of the second connector.

The first connector may have a pressure line that may be fluidly coupled to the aperture.

In a fourth aspect, there is provided an assembly comprising a first connector and a second connector that are configured to be assembled together to provide an inlet, an outlet, and an assembly gas flow passage; the first connector comprising a port; the second connector comprising a flow restriction configured to restrict flow through the gas flow passage, and an access passage configured to allow the port to be in fluid communication with the assembly gas flow passage.

The gas flow passage may be defined at least in part by a wall, and the access passage may comprise an aperture in the wall of the connector.

The flow restriction may be in a recess at the inlet of the second connector.

A portion and/or surface of the connector may be tapered.

The access passage may be arranged between a first sealing mechanism and the flow restriction.

The access passage may be provided at the flow restriction, or immediately adjacent and on a downstream side of the flow restriction.

The first sealing mechanism may comprise one or more of: a face seal, an O-ring, a lip seal, a wiper seal, or a sealing surface.

The sealing surface may comprise an arcuate surface.

The sealing surface may seal via a friction/interference fit with an internal surface of a second connector.

The connector may be a two part connector, a first part comprising the flow restriction and a second part comprising the first sealing mechanism.

The first and second sections may be linked.

The flow restriction may be upstream of the first sealing mechanism.

The assembly may further comprise a cavity defined by the first connector and the second connector.

The cavity may be defined by an external arcuate surface of the first connector.

The cavity may be in fluid communication with the gas flow passage via the access passage.

The cavity may have a longitudinal dimension that may be substantially parallel to a direction of gas flow in the gas flow passage.

The assembly may further comprise a second sealing mechanism arranged between the terminal end and the access passage and/or the cavity.

The second sealing mechanism may comprise one or more of: a face seal, an O-ring, a lip seal, a wiper seal, or a sealing surface.

The sealing surface may comprise an arcuate surface.

The sealing surface may seal via a friction/interference fit with an internal surface of a second connector.

The second connector further may comprise a stop.

The stop may be or may comprise a collar.

A surface of the collar may be configured to form a face seal with a surface of the second connector.

The first connector may have a pressure line that may be fluidly coupled to the aperture.

In a fifth aspect, there is provided a combination of a conduit and a connector according to any one of the first aspect or the second aspect.

The conduit may comprise a single use conduit.

The conduit and connector may be integral.

The conduit and connector may be separate components that are connectable together.

The conduit may be or may comprise a dry line or a conduit for directing a source of respiratory gas to a humidification chamber or for provision to a respiratory breathing circuit or system.

The conduit and connector may be adapted to provide gases at a flow rate of greater than or equal to about 5 or 10 litres per minute.

In a sixth aspect, there is provided a combination of a pressure relief valve and a connector according to the first aspect.

The pressure relief valve may be a reusable pressure relief valve.

The pressure relief valve and connector may be adapted to provide gases at a flow rate of greater than or equal to about 5 or 10 litres per minute.

In a seventh aspect, there is provided a respiratory gases system comprising a connector of any one of the first to fourth aspects, and a flow source adapted to provide gases at a flow rate of greater than or equal to about 5 or 10 litres per minute.

In an eighth aspect, there is provided a pressure relief device for use in a respiratory system comprising: a device inlet and a device outlet, a main gas flow passage between the device inlet and the device outlet, a pressure relief mechanism adapted to vent at least a portion of the gas flow when a pressure of the gas flow increases above a pressure threshold, and a sensing mechanism configured to dynamically adjust the pressure threshold. The outlet of pressure relief device is configured to receive a connector. An operating condition of the pressure relief device is determined by the connector and comprises one of the following operating configurations: (a) the sensing mechanism operates to dynamically adjust the pressure threshold based on a flow rate and/or pressure of the flow of gases through a portion of the pressure relief device or the respiratory system; (b) the sensing mechanism is inoperational and the pressure threshold comprises a set pressure threshold; (c) the pressure relief valve and sensing mechanism are inoperational and the pressure relief device delivers the gases flow to a patient without providing pressure relief.

The pressure relief device may further comprise a valve inlet in fluid communication with the device inlet, a vent outlet, a valve seat between the valve inlet and the vent outlet, and a valve member configured to seal against the valve seat and to displace from the valve seat by an inlet pressure at the valve inlet increasing above a pressure threshold to vent at least a portion of the flow of gases from the valve inlet to the vent outlet.

In an embodiment, the sensing mechanism comprises a sensing member configured to sense a differential pressure indicative of a flow rate and/or pressure of the flow of gases, a mechanical link configured to couple the sensing member and the valve member to transfer a force applied by the sensing member to the valve member to adjust a biasing of the valve member against the valve seat in response to the flow rate and/or pressure of the flow of gas.

In a further aspect, there is provided an assembly comprising the pressure relief device of the eighth aspect, and a connector. The connector is connected to the main outlet of the pressure relief device. The connector comprises an inlet end and an outlet end, a wall defining a connector gas flow passage between the inlet and outlet ends, a flow restriction, and an access passage through the wall. The sensing mechanism of the pressure relief device comprises a first sensing chamber in fluid communication with the flow of gases, upstream of the flow restriction such that the operating configuration of the pressure device is operating configuration (a).

In a further aspect, there is provided an assembly comprising the pressure relief device of the eighth aspect, and a connector, wherein the connector is connected to the main outlet of the pressure relief device. The connector comprises an inlet end and an outlet end, a wall defining a connector gas flow passage between the inlet and outlet ends; a flow restriction; and an access passage through the wall. The sensing mechanism of the pressure relief device comprises a first sensing chamber in fluid communication with the flow of gases, upstream of the flow restriction and a second sensing chamber in fluid communication of the flow of gases at or downstream of the flow restriction via the access passage, such that a resulting differential flow rate and/or pressure caused by the flow of gases through the flow restriction is sensed by the sensing member and the operating configuration of the pressure device is operating configuration (a).

The access passage may be positioned downstream of the flow restriction.

The access passage may be provided at the flow restriction, or immediately adjacent and on a downstream side of the flow restriction.

The flow restriction may be provided at or proximal to the inlet end.

In yet a further aspect, there is provided an assembly comprising the pressure relief device of the eighth aspect, and a connector, wherein the connector is connected to the main outlet of the pressure relief device. The connector comprises: an inlet end and an outlet end, a wall defining a connector gas flow passage between the inlet and outlet ends; and an access passage through the wall. The sensing mechanism of the pressure relief device comprises a first sensing chamber in fluid communication with the flow of gases, upstream of the connector and a second sensing chamber in fluid communication of the flow of gases via the access passage, such that a resulting differential flow rate and/or pressure is absent between the first sensing chamber and the second sensing chamber, and the operating configuration of the pressure device is operating configuration (b).

The pressure relief device may not include a flow restriction between the main inlet and the main outlet, and the connector may not include a flow restriction.

In an embodiment, the connector defines a gas flow passage having a substantially constant diameter.

The access passage may be substantially aligned with a communication line that is configured to fluidly connect the second sensing chamber to the flow of gases through the connector.

In yet a further aspect, there is provided an assembly comprising the pressure relief device of the eighth aspect, and a connector wherein the connector is connected to the main outlet of the pressure relief device. The connector comprises: an inlet end and an outlet end, and a wall defining a connector gas flow passage between the inlet and outlet ends. The sensing mechanism of the pressure relief device comprises a first sensing chamber in fluid communication with the flow of gases, upstream of the connector and a second sensing chamber that is blocked from being in fluid communication with the flow of gases via the wall of the connector, such that a resulting differential flow rate and/or pressure is absent between the first sensing chamber and the second sensing chamber and the operating configuration of the pressure device is operating configuration (c).

The connector may comprise an access passage through the wall, the access passage being misaligned with a communication line that is configured to fluidly connect the second sensing chamber to the flow of gases, such that the wall of the connector blocks the fluid communication between the sensing chamber and the flow of gases.

In a ninth aspect a pressure relief device for use in a respiratory system. The pressure relief device comprises: a device inlet and a device outlet; a main gas flow passage between the device inlet and the device outlet; and a pressure relief mechanism between the inlet and the outlet. The pressure relief mechanism comprises a substantially rigid valve connector portion configured to attach to a valve adjustment member; a valve diaphragm, a portion of the valve diaphragm being overmoulded to the valve connector portion; and a valve seat. The valve diaphragm and/or the valve connector portion is arranged to be seated against the valve seat in a first configuration; and to be spaced from the valve seat in a second configuration when pressure in the gas flow passage exceeds a pressure threshold.

The valve diaphragm and/or the valve connector portion may be adapted to seal against the valve seat in the first configuration.

In an embodiment, a tensile force in a portion of the valve diaphragm is greater in the second configuration than in the first configuration.

The device may comprise a valve frame, wherein a portion of the valve diaphragm is overmoulded to the valve frame.

In an embodiment, a portion of the valve diaphragm links the valve connector portion and the valve frame.

In an embodiment, a portion of the valve diaphragm between the valve connector portion and the valve frame is flexible.

The valve frame may be annular.

The valve frame may comprise one or both of engagement features and location features for attaching the valve frame to a body of the pressure relief device.

The valve connector portion may be positioned substantially centrally relative to the valve frame.

The device may comprise a sensing mechanism to dynamically adjust the pressure threshold based on a flow rate of a flow of gases through the outlet.

The sensing mechanism may comprise a sensing diaphragm and a substantially rigid sensing connector portion configured to attach to a valve adjustment member. A portion of the sensing diaphragm may be overmoulded to the sensing connector portion.

The sensing mechanism may comprise a sensing frame, with a portion of the sensing diaphragm is overmoulded to the sensing frame.

A portion of the sensing diaphragm may link the sensing connector portion and the sensing frame.

In an embodiment, a portion of the sensing diaphragm between the sensing connector portion and the sensing frame, is flexible.

The sensing frame may be annular.

The sensing frame may comprise one or both of engagement features and location features for attaching the sensing frame to a body of the pressure relief device.

The sensing connector portion may be positioned substantially centrally relative to the sensing frame.

The device may comprise a valve adjustment member, wherein the valve adjustment member comprises a mechanical link that links the sensing connector portion and the valve connector portion.

The sensing connector portion and the valve connector portion may each comprise an engagement feature for engaging with an end of the mechanical link.

The mechanical link may comprise a plurality of ribs.

The mechanical link may be located in a channel and slidable axially in the channel.

In an embodiment, the sensing connector portion, the valve connector portion, and the mechanical link are co-axial. The axis of the mechanical link may be substantially transverse to the general gas flow direction from the device inlet to the device outlet.

In an embodiment, the sensing connector portion and the valve connector portion each comprise a pair of spaced apart peripheral flanges. The flanges may be annular and co-axial, and each pair may define an annular space between the flanges.

In an embodiment, a portion of the valve diaphragm that is overmoulded to the valve connector portion is received into the annular space defined by the flanges on the valve connector portion.

In an embodiment, a portion of the sensing diaphragm that is overmoulded to the sensing connector portion is received into the annular space defined by the flanges on the sensing connector portion.

In an embodiment, a portion of the valve diaphragm and sensing diaphragm are in tension.

The valve diaphragm and/or sensing diaphragm may comprise elastomeric material.

The pressure relief mechanism and/or the sensing mechanism may comprise removable components.

The device may comprise a first sensing chamber of a first side of the sensing diaphragm, and a second sensing chamber on a second side of the sensing diaphragm, wherein the second sensing chamber is in fluid communication with a portion of a gas flow passage, downstream of a flow restriction or constriction, in the main gas flow passage between the device inlet and the device outlet or a gas flow passage in a respiratory system, optionally the fluid communication between the second sensing chamber and the portion of the gas flow passage is provided by a bleed line.

The device may comprise a first valve chamber on a side of the valve diaphragm opposite the side of the valve seat, the first valve chamber having an orifice in fluid communication with the atmosphere.

In an embodiment, the first valve chamber orifice comprises a filter and/or the communication line comprises a filter.

The filter may comprise a porous material.

The device may comprise a housing. The housing may comprise two or more parts screwed or ultrasonically welded together.

In a tenth aspect, there is provided a diaphragm component for use in a pressure relief device comprising: a flexible diaphragm and a substantially rigid connector portion configured to attach to a valve adjustment member. A portion of the diaphragm is overmoulded to the connector portion.

The connector portion may be adapted to removably attach to the valve adjustment member.

The valve adjustment member may comprise a mechanical link and the connector portion attaches to an end portion of the mechanical link.

In an embodiment, the connector portion comprises catches to engage with a peripheral surface of the end portion of the mechanical link. The catches may comprise protrusions that extend towards a central axis of the diaphragm member.

In an embodiment, the mechanical link comprises at least one recess, and the protrusions engage the recess(es). The at least one recess may comprise an annular recess.

In an embodiment, the connector portion comprises a boss.

In an embodiment, the connector portion comprises a pair of spaced apart peripheral flanges. The flanges may be annular and co-axial, and each pair may define an annular space between the flanges.

In an embodiment, a portion of the diaphragm that is overmoulded to the connector portion is received into the annular space defined by the flanges on the connector portion.

The diaphragm may be in tension and/or the diaphragm may comprise elastomeric material.

The diaphragm component may comprise a frame, wherein a portion of the diaphragm is overmoulded to the frame.

In an embodiment, a portion of the diaphragm links the connector portion and the frame. A portion of the diaphragm between the connector portion and the frame may be flexible.

The frame may be annular.

The connector portion is positioned substantially centrally relative to the frame.

The frame may comprise one or both of engagement features and location features for attaching the frame to a valve body of a pressure relief device.

In an eleventh aspect, there is provided a pressure relief device comprising: a device inlet and a device outlet, a main gas flow passage between the device inlet and the outlet, a pressure relief valve comprising a valve diaphragm adapted to vent at least a portion of a flow of gases through the gas flow passage when pressure in the gas flow passage exceeds a pressure threshold, and a sensing mechanism to dynamically adjust the pressure threshold based on a flow rate and/or pressure of the flow of gases through the gas flow passage. The sensing mechanism comprises a sensing diaphragm configured to sense a differential pressure indicative of a flow rate and/or pressure of the flow of gases. A mechanical link couples the sensing diaphragm to the valve diaphragm to transfer a force applied by the sensing diaphragm to the valve member to adjust a biasing of the valve member against the valve seat in response to the flow rate and/or pressure of the flow of gas. A damping arrangement is provided and configured to damp mechanical oscillations of the valve diaphragm and/or the sensing diaphragm, wherein at least a portion of the arrangement is configured to couple to the mechanical link.

The damping arrangement may comprise a guide channel for the mechanical link, the guide channel comprising a viscous fluid in contact with the mechanical link. The viscous fluid may seal between the guide channel and the mechanical link to prevent gas flow along the guide channel.

In an embodiment, the viscous fluid is a lubricant with high viscosity and low shear strength. For example, the viscous fluid may comprise a non-Newtonian fluid. Additionally or alternatively, the viscous fluid may demonstrate Bingham plastic and/or dilatant characteristics.

In an embodiment, the viscous fluid comprises grease.

The sensing mechanism may comprise a first sensing chamber in fluid communication with the main gas flow passage. Additionally, the damping arrangement may comprise a sealing boot to substantially seal against a portion of the mechanical link; and a passage providing fluid communication between a first sensing chamber of the sensing mechanism and the main gas flow passage. The passage may be defined by a damping aperture.

In an embodiment, the first sensing chamber is adjacent to the sensing diaphragm, wherein a wall of the sensing chamber comprises a link aperture through which the mechanical link passes.

In an embodiment, the sealing boot covers the link aperture to provide a seal between the mechanical link and the first sensing chamber wall.

The device may comprise a guide channel between the sensing diaphragm and the valve diaphragm, with the mechanical link being axially slidable in the channel. The sealing boot may be arranged at an end of the guide channel nearest the sensing diaphragm. Alternatively, the sealing boot may be arranged at an end of the guide channel nearest the valve diaphragm.

The device may comprise a retention mechanism to retain the sealing boot to the aperture or guide channel.

The sealing boot may define an aperture or channel to receive the mechanical link. The sealing boot may be flexible to allow axial movement of the mechanical link through a movement range.

In an embodiment, the sealing boot is curved. For example, the sealing boot may be convex relative to the first sensing chamber. Alternatively, the sealing boot may comprise a concertina membrane.

The sealing boot may allow the mechanical link to move axially through a movement range to provide a desired range of adjustment of the bias of the diaphragm. Preferably, the sealing boot provides negligible resistance to axial movement through the movement range.

In an embodiment, the sealing boot is resistant to buckling under axial loads sufficient to adjust the valve mechanism.

The sensing mechanism may comprise a first sensing chamber in fluid communication with the main gas flow passage, in which the damping arrangement comprises a magnetic arrangement to damp mechanical oscillations of pressure relief valve and/or the sensing mechanism.

The magnetic arrangement may comprise a conductive coil extending along a length of the mechanical link. The conductive coil may be electrically connected to an electrical resistor to dissipate heat.

In an embodiment, the magnetic arrangement further comprises a magnet arranged to induce an electrical current in the coil upon axial movement of the mechanical link.

The device may comprise a magnet in the form of a ring, arranged to surround the mechanical link.

In an embodiment, the magnetic arrangement comprises an electrically conductive member provided to the mechanical link. The electrically conductive member may be in the form of a ring.

The electromagnetic arrangement may further comprise first and second magnets fixed relative to a body of the pressure relief device. The first and second magnets may be ring magnets, arranged such that the mechanical link is axially movable within each ring. The first and second magnets are electromagnets or permanent magnets.

The magnetic arrangement may comprise a conductive coil surrounding the mechanical link and an electrically conductive member, the coil being fixed relative to a body of the pressure relief device. The coil may be electrically connected to an electrical resistor to dissipate heat.

The pressure relief valve may comprise a valve inlet in fluid communication with the device inlet, a vent outlet, a valve seat between the valve inlet and a vent outlet, and a valve diaphragm configured to seal against the valve seat and to displace from the valve seat by an inlet pressure at the valve inlet increasing above the pressure threshold to vent at least a portion of the flow of gases from the valve inlet to the vent outlet.

In a twelfth aspect, there is provided a pressure relief device comprising: a device inlet, a device outlet, a main gas flow passage between the inlet and the outlet; a pressure relief valve adapted to vent at least a portion of a flow of gases through the gas flow passage when pressure in the gas flow passage exceeds a pressure threshold, and a sensing mechanism to dynamically adjust the pressure threshold based on a flow rate and/or pressure of the flow of gases through the gas flow passage. The sensing mechanism comprises a mechanical link coupling the pressure relief valve and the sensing mechanism. The sensing mechanism comprises a first sensing chamber in fluid communication with the main gas flow passage, and a sealing boot to substantially seal against a portion of the mechanical link.

The pressure relief valve may comprise a valve diaphragm.

The sensing mechanism may comprise a sensing diaphragm.

The first sensing chamber may be adjacent to the sensing diaphragm, wherein a wall of the sensing chamber comprises a link aperture through which the mechanical link passes.

In an embodiment, the sealing boot extends across the aperture to provide a seal between the mechanical link and the sensing chamber wall.

The device may comprise a guide channel between the sensing diaphragm and the valve diaphragm, with the mechanical link being axially slidable in the channel. The guide channel may define the link aperture.

The sealing boot may be arranged at an end of the guide channel nearest the sensing diaphragm. Alternatively, the sealing boot may be arranged at an end of the guide channel nearest the valve diaphragm, or intermediate an end of the guide channel nearest the valve diaphragm and an end of the guide channel nearest the sensing diaphragm.

The device may comprise a retention mechanism to retain the sealing boot to the aperture or guide channel.

The sealing boot may define an aperture or channel to receive the mechanical link.

The sealing boot may seal around the mechanical link.

In an embodiment, the sealing boot is flexible to allow axial movement of the mechanical link through a movement range.

The sealing boot may be curved. For example, the sealing boot may be convex relative to the first sensing chamber. Alternatively, the sealing boot may comprise a concertina membrane. The sealing boot preferably provides negligible resistance to axial movement through the movement range and/or the sealing boot is resistant to buckling under axial loads sufficient to adjust the valve mechanism.

The sealing boot may allow the mechanical link to move axially through a movement range to provide a desired range of adjustment of the bias of the diaphragm.

The sensing mechanism may comprise a sensing diaphragm, the first sensing chamber being adjacent to the sensing diaphragm. A wall of the first sensing chamber further comprising a damping aperture in fluid communication between the first sensing chamber and the main gas flow passage.

The wall of the first sensing chamber may comprise a plurality of damping apertures.

The sensing mechanism comprising a second sensing chamber on an opposite side of the sensing diaphragm to the first sensing chamber, wherein the second sensing chamber is in fluid communication with a portion of a gas flow passage downstream of a flow restriction/constriction, in the main gas flow passage between the device inlet and the device outlet or a gas flow passage in a respiratory system, optionally the fluid communication between the second sensing chamber and the portion of the gas flow passage is provided by a communication line.

A valve seat may be positioned on one side of the valve diaphragm and a valve chamber on the opposite side. One side of the valve diaphragm may be configured to seal against the valve seat, with the valve chamber being in fluid communication with the atmosphere via an orifice.

The orifice may comprise a filter and/or the communication line may comprise a filter. The filter may comprise a porous material.

The device may comprise a body defining the device inlet and outlet.

The device may comprise two caps configured to cooperate to house the pressure relief device, the two caps being screwed or ultrasonically welded together.

In a thirteenth aspect, there is provided a pressure relief device comprising: a device inlet, a device outlet, a gas flow passage between the device inlet and the outlet, a pressure relief valve adapted to vent at least a portion of a flow of gases through the gas flow passage when pressure in the gas flow passage exceeds a pressure threshold, and a sensing mechanism to dynamically adjust the pressure threshold based on a flow rate and/or pressure of the flow of gases through the gas flow passage. The sensing mechanism comprises a viscous fluid to damp mechanical oscillations of the pressure relief valve and/or of the sensing mechanism and wherein the sensing mechanism comprises a first sensing chamber in fluid communication with the main gas flow passage.

The pressure relief valve may comprise a valve diaphragm and/or the sensing mechanism comprises a sensing diaphragm. In an embodiment, the device comprises a mechanical link coupling the valve diaphragm and the sensing diaphragm.

The device may comprise a guide channel between the sensing diaphragm and the valve diaphragm, with the mechanical link being axially slidable in the channel.

In an embodiment, the viscous fluid is provided in the guide channel to damp movement of the mechanical link. The viscous fluid may seal between the guide channel and the mechanical link to prevent gas flow along the guide channel. The viscous fluid may comprise a lubricant with high viscosity and low shear strength. For example, the viscous fluid may comprise a non-Newtonian fluid and/or demonstrate Bingham plastic and/or dilatant characteristics. For example, the viscous fluid may comprise grease.

The first sensing chamber may comprise a damping aperture to provide for fluid communication between the first sensing chamber and the main gas flow passage. The first sensing chamber may comprise a plurality of damping apertures.

The sensing mechanism may comprise a second sensing chamber on an opposite side of the sensing diaphragm to the first sensing chamber. The second sensing chamber may be in fluid communication with a portion of a gas flow passage downstream of a flow restriction/constriction, in the main gas flow passage between the device inlet and device outlet or a gas flow passage in a respiratory system. Optionally the fluid communication between the second sensing chamber and the portion of the gas flow passage is provided by a communication line.

The device may comprise a valve seat positioned on one side of the valve diaphragm and a valve chamber on the opposite side. One side of the valve diaphragm may be configured to seal against the valve seat, and the valve chamber may be in fluid communication with the atmosphere via an orifice.

The orifice may comprise a filter and/or the communication line may comprise a filter. The filter may comprise a porous material.

The device may comprise a valve body defining the device inlet and outlet. The device may comprise two caps configured to cooperate to house the pressure relief device, the caps being screwed or ultrasonically welded together.

In a fourteenth aspect, there is provided a pressure relief device comprising: an inlet, an outlet, a gas flow passage between the inlet and the outlet, a pressure relief valve adapted to vent at least a portion of a flow of gases through the gas flow passage when pressure in the gas flow passage exceeds a pressure threshold, a sensing mechanism to dynamically adjust the pressure threshold based on a flow rate and/or pressure of the flow of gases through the gas flow passage, and a magnetic arrangement to damp mechanical oscillations of pressure relief valve and/or the sensing mechanism.

The pressure relief valve may comprise a valve diaphragm and/or the sensing mechanism comprises a sensing diaphragm. A mechanical link may couple the pressure relief valve and the sensing mechanism. A guide channel or guide aperture may be provided between the sensing diaphragm and the valve diaphragm, with the mechanical link being axially slidable in the guide channel.

In an embodiment, the magnetic arrangement may comprise a conductive coil extending along a length of the mechanical link. The conductive coil may be electrically connected to an electrical resistor to dissipate heat.

The magnetic arrangement may further comprises a magnet arranged to induce an electrical current in the coil upon axial movement of the mechanical link. The magnet may be in the form of a ring that surrounds the mechanical link.

The magnetic arrangement may comprise an electrically conductive member provided to the mechanical link. The electrically conductive member may be in the form of a ring.

In an embodiment, the magnetic arrangement further comprises first and second magnets fixed relative to a body of the pressure relief device. The first and second magnets may comprise ring magnets, arranged such that the mechanical link is axially movable within each ring. The first and second magnets may be electromagnets or permanent magnets.

The magnetic arrangement may comprise a conductive coil surrounding the mechanical link and an electrically conductive member, the coil being fixed relative to a body of the pressure relief device. The coil may be electrically connected to an electrical resistor to dissipate heat.

The device may comprise a sensing chamber on an opposite side to of the sensing diaphragm to the mechanical member, wherein the sensing chamber is in fluid communication with a portion of a gas flow passage downstream of a flow restriction/constriction, in the main gas flow passage between the device inlet and the device outlet or a gas flow passage in a respiratory system. Optionally the fluid communication between the second sensing chamber and the portion of the gas flow passage may be provided by a communication line.

The device may comprise a valve seat positioned on one side of the valve diaphragm and a valve chamber on the opposite side, wherein one side of the valve diaphragm is configured to seal against the valve seat; and wherein the valve chamber is in fluid communication with the atmosphere via an orifice.

The orifice may comprise a filter and/or the communication line may comprise a filter. The filter comprises a porous material.

The device may comprise a housing. The housing may comprise two or more parts screwed or ultrasonically welded together.

In a fifteenth aspect, there is provided a respiratory system comprising a flow source, a pressure relief device as described above, and a patient interface. Gas from the flow source flows to the patient interface via the pressure relief device.

In an embodiment, the system comprises a humidifier positioned between the flow source and the patient interface. The humidifier may be positioned downstream of the pressure relief device, with a conduit connecting the outlet of the pressure relief device to an inlet of the humidifier.

An inspiratory conduit may be provided between the humidifier and the patient interface.

The pressure relief device may be coupled to the flow source.

In use, the pressure relief device may be oriented such that the diaphragms are substantially vertical with respect to a ground surface.

The pressure relief device may comprise a flange at the device inlet.

A connector as described above may be provided at the outlet of the pressure relief device.

In an embodiment, the patient interface comprises a nasal cannula. The cannula may comprise a non-sealing nasal cannula. The nasal cannula may be switchable between two configurations, wherein in a first configuration, the nasal cannula delivers a first flow rate of gases to a patient, and in a second configuration, the nasal cannula delivers a second flow rate of gases to the patient, wherein the first and second flow rates are different.

The second flow rate may be lower than the first flow rate.

The second flow rate may comprise substantially no flow to the nasal cannula.

The terms ‘conduit’ and ‘tubing’ as used in this specification and claims are intended to broadly mean, unless the context suggests otherwise, any member that forms or provides a lumen for directing a flow of liquid or gases. For example, a conduit or conduit portion may be part of a humidification device, or may be a separate conduit attachable to a humidification device to provide a flow of fluid or a fluid communication.

The terms ‘comprising’ and/or ‘including’ as used in this specification and claims means ‘consisting at least in part of’. When interpreting each statement in this specification and claims that includes the term ‘comprising’ and/or ‘including’, features other than that or those prefaced by the term may also be present. Related terms such as ‘comprise’ and ‘comprises’, ‘include’ and ‘includes’ are to be interpreted in the same manner.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

As used herein the term ‘and/or’ means ‘and’ or ‘or’, or both.

As used herein ‘(s)’ following a noun means the plural and/or singular forms of the noun.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

The disclosure consists in the foregoing and also envisages constructions of which the following gives examples only. Features disclosed herein may be combined into new embodiments of compatible components addressing the same or related inventive concepts.

### BRIEF DESCRIPTION OF THE FIGURES

Preferred embodiments of the disclosure will be described by way of example only and with reference to the following drawings.

Specific embodiments and modifications thereof will become apparent to those skilled in the art from the detailed description herein having reference to the figures that follow, of which:

FIG. 1A illustrates a high flow respiratory system.

FIG. 1B is a schematic representation of a flow controlled pressure relief valve.

FIG. 1C is a perspective view of one embodiment of the connector and a flow controlled pressure relief or pressure regulating device.

FIG. 2 is a cross-sectional view of the connector and a flow controlled pressure relief or pressure regulating device of FIG. 1C.

FIG. 3 is a perspective view of the connector of FIG. 2.

FIG. 4 is a cross-sectional view of another embodiment connector and a flow controlled pressure relief or pressure regulating device.

FIG. 5 is a perspective view of the connector of FIG. 4.

FIG. 6 is a cross-sectional view of another embodiment connector and a flow controlled pressure relief or pressure regulating device.

FIG. 7 is a perspective view of the connector of FIG. 6.

FIG. 8 is a cross-sectional view of another embodiment connector and a flow controlled pressure relief or pressure regulating device.

FIG. 9 is a cross-sectional view of the connector of FIG. 7 and one variation of a second connector.

FIG. 10 is a cross-sectional view of the connector of FIG. 7 and another variation of a second connector.

FIG. 11 is a perspective cross-sectional view of the connector and the second connector of FIG. 10.

FIG. 12 is a cross-sectional view of the connector of FIG. 7.

FIG. 13 is a cross-sectional view of another embodiment connector and a flow controlled pressure relief or pressure regulating device.

FIG. 14 is a cross-sectional view of another embodiment connector and a flow controlled pressure relief or pressure regulating device.

FIG. 15 is a schematic cross-sectional view of another embodiment connector and a flow controlled pressure relief or pressure regulating device.

FIG. 16 is a cross-sectional view of another embodiment connector and a flow controlled pressure relief or pressure regulating device.

FIG. 17 is a cross-sectional view of another embodiment connector and a flow controlled pressure relief or pressure regulating device.

FIG. 18 is a cross-sectional view of another embodiment connector and a flow controlled pressure relief or pressure regulating device.

FIG. 19 is a cross-sectional view of another embodiment connector and a flow controlled pressure relief or pressure regulating device.

FIG. 20 illustrates a tuning routine for a FCPRV.

FIG. 21 is a perspective view of a further embodiment connector.

FIG. 22 is a side elevation view of the connector of FIG. 21.

FIG. 23 is a section view of the connector of FIGS. 21 and 22, taken through a centreline of the connector.

FIG. 24 is a section view of one embodiment pressure relief device having two diaphragm components.

FIG. 25 is a top perspective view of one embodiment diaphragm component for use in a pressure relief device such as the one shown in FIG. 24.

FIG. 26 is a bottom perspective view of the diaphragm component of FIG. 21.

FIG. 27 is a side section view taken through line A-A of FIG. 26.

FIG. 28 is a side section view taken through line A-A of FIG. 26, but only showing the flexible diaphragm of the diaphragm component, with the frame and link connector hidden.

FIG. 29A is a bottom plan view of the diaphragm component.

FIG. 29B is a top plan view of the diaphragm component of FIG. 29A.

FIG. 30 is a perspective view of one embodiment pressure relief device, with the valve chamber caps and coupler hidden.

FIG. 31 is a side section view of the pressure relief device of FIG. 30, with the section taken along a centreline of the device.

FIG. 32 is a detail perspective view of a portion of FIG. 31, showing the damping orifice and sealing boot.

FIG. 33A is a perspective view of the sealing boot of FIGS. 31 and 32.

FIG. 33B is a side section view taken through a centreline of the sealing boot of FIG. 33A.

FIG. 34 is a perspective section view of a further embodiment pressure relief valve, with the valve chamber caps hidden and the section taken along a centreline of the device.

FIG. 35 is a side section view corresponding to FIG. 30.

FIG. 36 is a schematic view of an arrangement for electromagnetically damping movement of the mechanical link, in which the mechanical link comprises a conductive coil.

FIG. 37 is a schematic view of a further arrangement for electromagnetically damping movement of the mechanical link, in which the mechanical link comprises a conductive ring.

FIG. 38 is a perspective view of the pressure relief valve of FIG. 25, showing the valve chamber caps.

FIG. 39 is a perspective of one of the valve chamber caps of FIG. 38, showing the fastener holes for joining two chamber caps together.

FIG. 40 is an illustrative perspective view of the pressure relief valve of FIG. 33 in a vertical orientation in use, with the inlet coupled to a gas supply, and the outlet.

FIG. 41 is a side section view of a pressure relief device having an alternative sealing boot and damping aperture arrangement, with the section taken along a centreline of the device.

FIG. 42 is a detail view of the detail F42 of FIG. 41.

FIG. 43 is a side section view of a further pressure relief device having a further alternative sealing boot and damping aperture arrangement, with the section taken along a centreline of the device.

FIG. 44 is a detail view of the detail F44 of FIG. 43.

FIG. 45 is a side section view of a further pressure relief device having a connector with sensing apertures provided immediately adjacent and downstream of the flow restriction.

FIG. 46 is a perspective section view corresponding to FIG. 45.

FIG. 47 is a side section view of a further pressure relief device having a connector with sensing apertures provided at the flow restriction.

FIG. 48 is a perspective section view corresponding to FIG. 47.

FIG. 49 is a partial perspective view showing the connection between the connector portion of a further embodiment valve member and one end of a mechanical link.

FIG. 50 is a partial section view through the connector portion and mechanical link of FIG. 49.

### DETAILED DESCRIPTION

Various embodiments are described with reference to the figures. Throughout the figures and specification, similar reference numerals may be used to designate the same or similar components, and redundant descriptions thereof may be omitted.

A connector according to embodiments described herein is particularly adapted for use in respiratory systems such as CPAP or high flow respiratory gas systems, for example a high flow system for use in anaesthesia procedures. Respiratory systems in which the connector may be particularly useful are CPAP, BiPAP, high flow therapy, varying high flow therapy, low flow air, low flow O₂ delivery, bubble CPAP, apnoeic high flow (i.e. high flow to anesthetized patients), invasive ventilation and non-invasive ventilation. Further, a connector as described herein may be useful in systems other than respiratory systems. A connector according to embodiments described herein is particularly adapted for use with a pressure relief or regulating device.

Unless the context suggests otherwise, a flow source provides a flow of gases at a set flow rate. A set flow rate may be a constant flow rate, variable flow rate or may be an oscillating flow rate, for example a sinusoidal flow rate or a flow rate with a step or square wave profile. Unless the context suggests otherwise a pressure source provides a flow of gases at a set pressure. The set pressure may be a constant pressure, variable pressure or may be an oscillating pressure, for example a sinusoidal pressure or a pressure with a step or square wave profile.

‘High flow therapy’ as used in this disclosure may refer to delivery of gases to a patient at a flow rate of greater than or equal to about 5 or 10 litres per minute (5 or 10 LPM or L/min).

In some configurations, ‘high flow therapy’ may refer to the delivery of gases to a patient at a flow rate of about 5 or 10 LPM to about 150 LPM, or about 15 LPM to about 95 LPM, or about 20 LPM to about 90 LPM, or about 25 LPM to about 85 LPM, or about 30 LPM to about 80 LPM, or about 35 LPM to about 75 LPM, or about 40 LPM to about 70 LPM, or about 45 LPM to about 65 LPM, or about 50 LPM to about 60 LPM. For example, according to those various embodiments and configurations described herein, a flow rate of gases supplied or provided to an interface via a system or from a flow source, may comprise, but is not limited to, flows of at least about 5, 10, 20, 30, 40, 50, 60, 70, 80, 90, 100, 110, 120, 130, 140, 150 LPM, or more, and useful ranges may be selected to be any of these values (for example, about 20 LPM to about 90 LPM,bout 40 LPM to about 70 LPM, about 40 LPM to about 80 LPM, about 50 LPM to about 80 LPM, about 60 LPM to about 80 LPM, about 70 LPM to about 100 LPM, about 70 LPM to about 80 LPM).

The gas delivered will be chosen depending on the intended use of the therapy. Gases delivered may comprise a percentage of oxygen. In some configurations, the percentage of oxygen in the gases delivered may be about 15% to about 100%, 20% to about 100%, or about 30% to about 100%, or about 40% to about 100%, or about 50% to about 100%, or about 60% to about 100%, or about 70% to about 100%, or about 80% to about 100%, or about 90% to about 100%, or about 100%, or 100%.

In some embodiments, gases delivered may comprise a percentage of carbon dioxide. In some configurations, the percentage of carbon dioxide in the gases delivered may be more than 0%, about 0.3% to about 100%, about 1% to about 100%, about 5% to about 100%, about 10% to about 100%, about 20% to about 100%, or about 30% to about 100%, or about 40% to about 100%, or about 50% to about 100%, or about 60% to about 100%, or about 70% to about 100%, or about 80% to about 100%, or about 90% to about 100%, or about 100%, or 100%.

High flow therapy has been found effective in meeting or exceeding the patient's normal real inspiratory demand, to increase oxygenation of the patient and/or reduce the work of breathing. Additionally, high flow therapy may generate a flushing effect in the nasopharynx such that the anatomical dead space of the upper airways is flushed by the high incoming gas flows. This creates a reservoir of fresh gas available of each and every breath, while minimising re-breathing of carbon dioxide, nitrogen, etc.

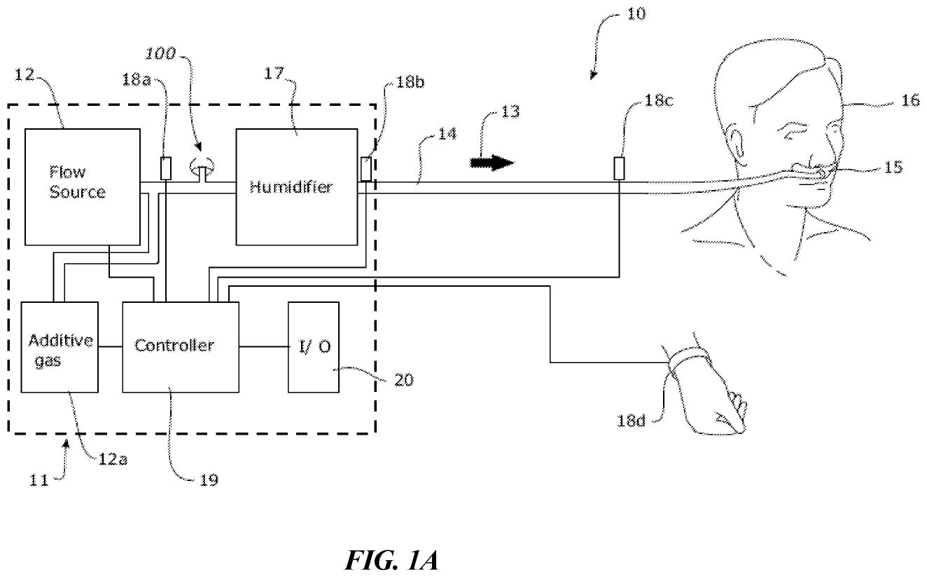

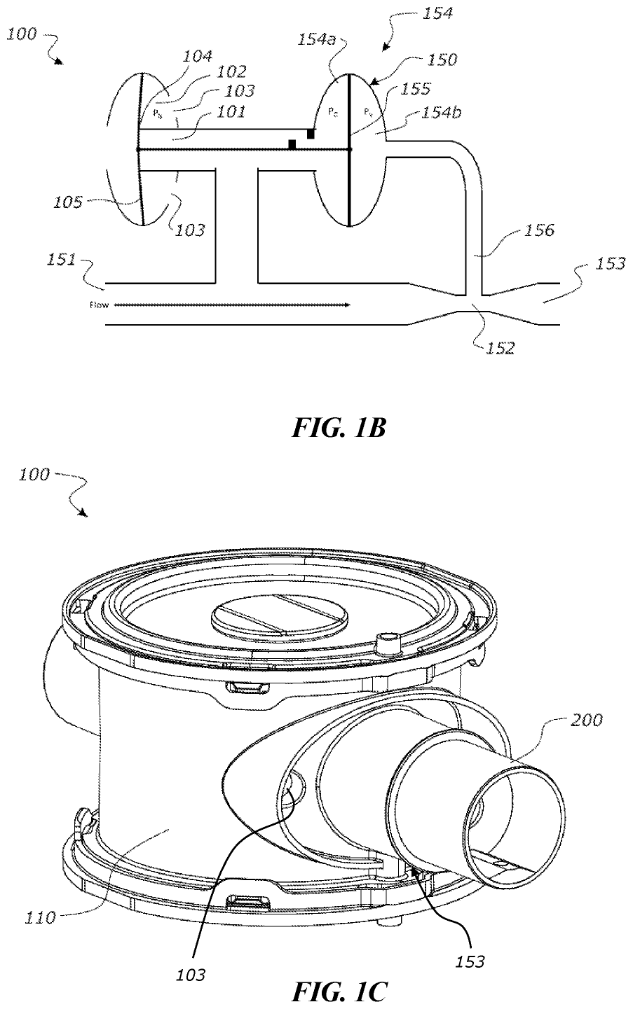

By example, a high flow respiratory system 10 is described with reference to FIG. 1A. High flow therapy may be used as a means to promote gas exchange and/or respiratory support through the delivery of oxygen and/or other gases, and through the removal of CO2 from the patient's airways. High flow therapy may be particularly useful prior to, during or after a medical procedure.

When used prior to a medical procedure, high gas flow can pre-load the patient with oxygen so that their blood oxygen saturation level and volume of oxygen in the lungs is higher to provide an oxygen buffer while the patient is in an apnoeic phase during the medical procedure.

A continuous supply of oxygen is essential to sustain healthy respiratory function during medical procedures (such as during anaesthesia) where respiratory function might be compromised (e.g. diminishes or stops). When this supply is compromised, hypoxia and/or hypercapnia can occur. During medical procedures such as anaesthesia and/or general anaesthesia where the patient is unconscious, the patient is monitored to detect when this happens. If oxygen supply and/or CO₂ removal is compromised, the clinician stops the medical procedure and facilitates oxygen supply and/or CO₂ removal. This can be achieved for example by manually ventilating the patient through an anaesthetic bag and mask, or by providing a high flow of gases to the patient's airway using a high flow therapy system.

Further advantages of high gas flow can include that the high gas flow increases pressure in the airways of the patient, thereby providing pressure support that opens airways, the trachea, lungs/alveolar and bronchioles. The opening of these structures enhances oxygenation, and to some extent assists in removal of CO₂.

The increased pressure can also keep structures such as the larynx from blocking the view of the vocal chords during intubation. When humidified, the high gas flow can also prevent airways from drying out, mitigating mucociliary damage, and reducing risk of laryngospasms and risks associated with airway drying such as nose bleeding, aspiration (as a result of nose bleeding), and airway obstruction, swelling and bleeding. Another advantage of high gas flow is that the flow can clear smoke created during surgery in the air passages. For example, smoke can be created by lasers and/or cauterizing devices.

A pressure relief or regulating device is particularly desirable for use in a respiratory system such as a high flow system comprising an unsealed patient interface, to provide an upper pressure limit for the system. Most importantly, the upper pressure limit may be configured to provide a patient safety limit, or may be configured to prevent damage to tubes, fluid connections, or other components. A pressure relief or regulating device may be used in a sealed system, such as CPAP (continuous positive airway pressure), BiPAP (bilevel positive airway pressure) and/or Bubble CPAP systems to regulate the pressure provided to the patient.

With reference to FIG. 1A, the system/apparatus 10 may comprise an integrated or separate component based arrangement, generally shown in the dotted box 11 in FIG. 1A. In some configurations the system 10 could comprise a modular arrangement of components. Hereinafter the system/apparatus 10 will be referred to as system, but this should not be considered limiting. The system 10 may include a flow source 12, such as an in-wall source of oxygen, an oxygen tank, a blower, a flow therapy apparatus, or any other source of oxygen or other gas. The system 10 may also comprise an additive gas source 12a, comprising one or more other gases that can be combined with the flow source 12. The flow source 12 can provide a pressurised high gas flow 13 that can be delivered to a patient 16 via a delivery conduit 14, and patient interface 15 (such as a nasal cannula). A controller 19 controls the flow source 12 and additive gas source 12a through valves or the like to control flow and other characteristics such as any one or more of pressure, composition, concentration, volume of the high flow gas 13. A humidifier 17 is also optionally provided, which can humidify the gas under control of the controller and control the temperature of the gas. One or more sensors 18a, 18b, 18c, 18d, such as flow, oxygen, pressure, humidity, temperature or other sensors can be placed throughout the system and/or at, on or near the patient 16. The sensors can include a pulse oximeter 18d on the patient for determining the oxygen concentration in the blood.

The controller 19 may be coupled to the flow source 12, the additive gas source 12a, humidifier 17 and sensors 18a-18d. The controller 19 can operate the flow source to provide the delivered flow of gas. It can control the flow, pressure, composition (where more than one gas is being provided), volume and/or other parameters of gas provided by the flow source based on feedback from sensors. The controller 19 can also control any other suitable parameters of the flow source to meet oxygenation requirements. The controller 19 can also control the humidifier 17 based on feedback from the sensors 18a-18d. Using input from the sensors, the controller can determine oxygenation requirements and control parameters of the flow source 12 and/or humidifier 17 as required. An input/output (I/O) interface 20 (such as a display and/or input device) is provided. The input device is for receiving information from a user (e.g. clinician or patient) that can be used for determining oxygenation requirements. In some embodiments, the system may be without a controller and/or I/O interface. A medical professional such as a nurse or technician may provide the necessary control function.

The pressure may also be controlled. As noted above, the high gas flow (optionally humidified) can be delivered to the patient 16 via a delivery conduit 14 and the patient interface 15 or ‘interface’, such as a cannula, mask, nasal interface, oral device or combination thereof. In some embodiments, the high gas flow (optionally humidified) can be delivered to the patient 16 for surgical uses, e.g. surgical insufflation. In these embodiments, the ‘interface’ could be a surgical cannula, trocar, or other suitable interface. The patient interface can be substantially sealed, partially sealed or substantially unsealed. A nasal interface as used herein is a device such as a cannula, a nasal mask, nasal pillows, or other type of nasal device or combinations thereof. A nasal interface can also be used in combination with a mask or oral device (such as a tube inserted into the mouth) and/or a mask or oral device (such as a tube inserted into the mouth) that can be detached and/or attached to the nasal interface. A nasal cannula is a nasal interface that includes one or more prongs that are configured to be inserted into a patient's nasal passages. A mask refers to an interface that covers a patient's nasal passages and/or mouth and can also include devices in which portions of the mask that cover the patient's mouth are removable, or other patient interfaces such as laryngeal mask airway or endotracheal tube. A mask also refers to a nasal interface that includes nasal pillows that create a substantial seal with the patient's nostrils. The controller controls the system to provide the required oxygenation.

A system 10 according to embodiments herein includes a pressure relief or regulating device, or pressure limiting device 100 (herein a pressure relief valve or PRV). The pressure limiting device 100 may be a valve having features described in WO/2018/033863, the entirety of which is hereby incorporated by reference herein. The connector may be used with other valves and/or devices. The PRV may be placed anywhere in the system between the flow source 12 and the patient 16. Preferably, the PRV 100 is provided at an outlet of the flow source 12, or between the flow source 12 and the humidifier 17, for example near to an inlet of the humidifier 17. In some embodiments, the PRV 100 may be provided at an outlet of the humidifier 17 and/or an inlet to the conduit 14, or at any point along the conduit 14 through a suitable housing or coupling device. The PRV 100 may be located anywhere in the system, for example the PRV could be part of the patient interface 15. The system may additionally or alternatively include a flow controlled pressure relief or pressure regulating device (FCPRV).

A PRV 100 according to the present disclosure regulates pressure at an approximately consistent pressure across a given range of flow rates. The PRV 100 may be used to provide an upper limit for patient safety, and/or to prevent damage to system components caused by overpressure. For example, an occlusion in the system may cause a substantial back pressure in the system upstream of the occlusion, and the PRV may operate to ensure the back pressure does not increase above a limit to protect the patient and/or system components from damage. A blockage in the patient's nares or exhaling conduit can result in an increased patient pressure. An occlusion in the system may be caused by for example inadvertent folding or crushing of the conduit 14, or may be caused deliberately, for example by occluding the conduit 14 (e.g. by pinching a portion of the conduit closed) to prevent a flow of gases from reaching the patient.

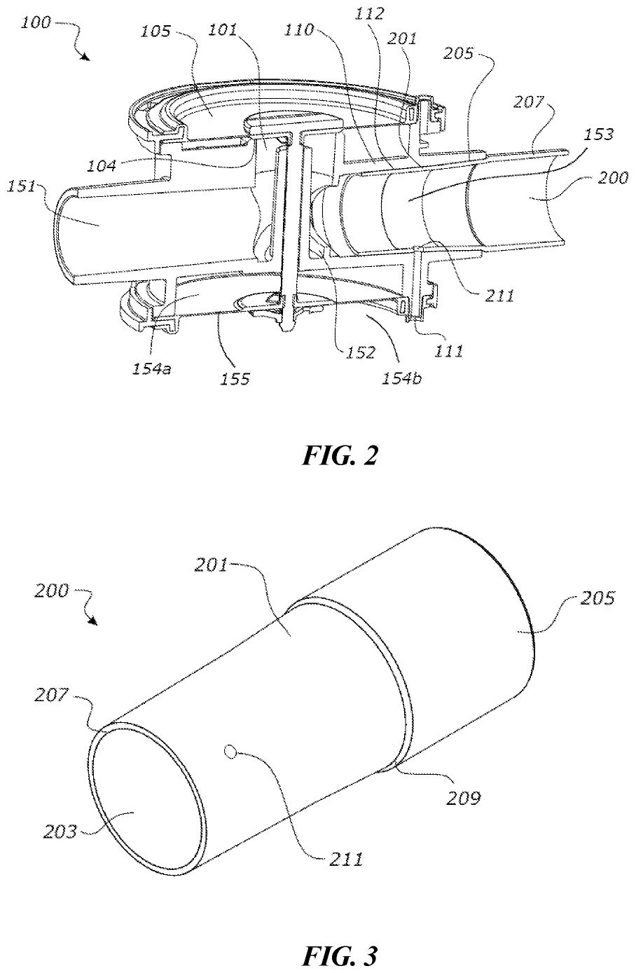

FIGS. 1C and 2 show one embodiment PRV included in a flow controlled pressure regulating valve (FCPRV) 100, which is illustrated schematically in FIG. 1B. The PRV comprises an inlet 101, an outlet chamber 102 with an outlet 103, a valve seat 104 between the inlet 101 and the outlet chamber 102, and a valve member 105 biased to seal against the valve seat 104. The valve member 105 is adapted to displace from the valve seat by pressure Pc at the PRV inlet 101 increasing above a pressure threshold. The pressure Pc acts on the valve member 105 to force the member away from the valve seat 104 once the pressure Pc reaches or exceeds the threshold. As the valve member 105 displaces from the valve seat 104, a flow of gases flows from the inlet 101 into the outlet chamber 102, and then from the outlet chamber 102 via the outlet 103 to ambient pressure/atmospheric pressure. The outlet from the chamber is configured so that the flow of gases through the outlet causes a (back) pressure Pb in the outlet chamber that acts on the valve member 105 to further displace the valve member 105 from the valve seat 104. As the valve member 105 is further displaced from the valve seat 104, a gap between the valve member 105 and valve seat 104 increases.

The FCPRV 100 further comprises a sensing mechanism 150 to dynamically adjust the pressure threshold at which the PRV 100 vents pressure based on the flow rate and/or pressure of the gases or a portion thereof, passing through the FCPRV or through the respiratory system. In certain embodiments, the FCPRV 100 comprises a sensing mechanism 150 to dynamically adjust the pressure threshold at which the PRV 100 vents pressure based on the flow rate of the gases or a portion thereof, passing through the FCPRV or through the respiratory system. In certain embodiments, the FCPRV 100 comprises a sensing mechanism 150 to dynamically adjust the pressure threshold at which the PRV 100 vents pressure based on the pressure of the gases or a portion thereof, passing through the FCPRV or through the respiratory system. A connector 200 according to embodiments described herein can be used with the FCPRV 100.

Referring to FIGS. 1B and 2, features and functionality of the FCPRV will now be described. The FCPRV 100 comprises a body 110 defining a main inlet 151 and a main outlet 153. In the illustrated embodiment, the sensing mechanism 150 includes a flow restriction or flow constriction 152 between the main inlet 151 and main outlet 153 of the FCPRV. The main inlet 151 and/or main outlet 153 are preferably integral with or defined by the FCPRV body 110. In the embodiment of FIGS. 1B and 2, the flow restriction 152 is part of the FCPRV body. In later described embodiments, the flow restriction is part of the connector. For ease of reference, the term ‘flow restriction’ may be used herein to describe both a flow restriction such as an orifice plate and a flow constriction such as used in a venturi. In operation, the flow of gases in a respiratory system flow through the FCPRV 100 from the main inlet 151 to the main outlet 153. The sensing mechanism 150 senses the flow rate/pressure of gases flowing to the patient at or downstream of the flow restriction/ constriction. In the embodiment shown, the pressure relief valve inlet 101 is between the FCPRV main inlet 151 and main outlet 153, and the flow restriction/constriction is downstream of the PRV inlet, but upstream of the main outlet 153. The sensing mechanism 150 senses the flow rate and/or pressure of gases flowing to the patient at or through the main outlet 153 of the valve.

The sensing mechanism 150 also includes a sensing chamber 154, and a sensing member 155 located in the sensing chamber 154. The sensing member 155 divides the sensing chamber 154 into a first chamber 154a and a second chamber 154b. The first chamber 154a is in fluid communication with the flow of gases upstream of the flow restriction 152, e.g. the first chamber 154a is in fluid communication with the main inlet 151 and the valve inlet 101 upstream of the restriction 152. The second chamber 154b is in fluid communication with the flow of gases at the flow constriction 152 or downstream of the flow restriction 152. In some embodiments, the device comprises a flow constriction configured as a venturi, with the second chamber 154b in fluid communication with the constriction via a pressure ‘tap’ or communication line 156 (FIG. 1B). However in an alternative configuration the device may comprise a flow restriction 152, e.g. an orifice plate, and the first and second chambers may tap off either side of the orifice plate, for example via pressure ‘tap’ or communication line 111 shown in FIG. 2. A pressure differential may be generated in any other suitable way, for example by a permeable membrane or a filter with a known pressure drop (a flow restriction).

A resulting pressure drop caused by the flow of gases that pass from the main inlet 151 to the main outlet 153 of the device, through the restriction 152 is therefore sensed by the sensing member 155 located within the sensing chamber 154.

In order to increase the flow rate through the respiratory system, the pressure provided by the flow source 12 is increased, increasing the pressure at the main inlet 151 and also in the first chamber 154a of the sensing chamber 154. As the flow rate through the FCPRV increases, a larger pressure drop is created by the restriction 152 due to an increased velocity of the gases passing through the restriction 152, and the pressure Pᵥ in the second chamber 154b of the sensing chamber 154 decreases. Thus an increasing flow rate through the FCPRV 100 from the main inlet 151 to the main outlet 153 results in an increasing differential pressure across the sensing member 155, with the first chamber 154a being a high (higher) pressure side of the sensing chamber 154 and the second chamber 154b being a low (lower) pressure side of the sensing chamber 154. This causes the sensing member 155 to move towards the low pressure side of the sensing chamber 154, away from the PRV valve member 105.

The sensing member 155 is mechanically coupled to the valve member 105 of the pressure relief valve 100, so that as the sensing member 155 moves towards the lower pressure side of the sensing chamber 154, the sensing member 155 pulls or biases the valve member 105 of the PRV against the valve seat 104. For a given flow rate setting, a higher flow rate causes a higher differential pressure across the sensing member 155, biasing the valve member 105 further towards the valve seat 104. This causes the pressure relief threshold for the PRV to increase. If a flow restriction (e.g. squashed conduit 14 or blockage in patient's nare) is introduced, the flow source 12 (rapidly) adjusts to increase pressure in the system to maintain the flow rate at a desired level. If the system pressure required to maintain the desired flow rate is above the relief pressure, the PRV begins to vent, with a portion of the flow provided to the main inlet 151 venting via the PRV valve member 105, and a portion of the flow passing through the restriction 152 and from the main outlet 153. The flow source 12 maintains a set flow rate to the main inlet 151 of the FCPRV 100. Thus as the PRV begins to vent, the flow rate through the constriction or restriction 152 decreases, and the pressure differential acting on the sensing member 155 decreases. This causes the bias provided by the sensing member 155 to the valve member 105 to decrease, and therefore the pressure relief threshold for the PRV 100 to decrease. In an ideal situation, an equilibrium state will be reached, whereby the patient receives as much flow as possible without exceeding the pressure relief threshold, or without exceeding a maximum delivered pressure at the patient interface.

If the flow restriction completely (or substantially completely) blocks the system, for example a conduit 14 is completely occluded (complete crushed or pinched closed) or a patient's nare is completely blocked, all or substantially all flow delivered to the main inlet 151 of the FCPRV 100 is vented via the PRV valve member 105.

FIG. 1B shows a body that provides or forms the outlet chamber 102 and the first chamber 154a of the sensing chamber 154. Those features are not shown in the other figures, but it will be appreciated that any of the embodiments of the PRV, FCPRC, or connector described herein may be used with a valve body having those features.

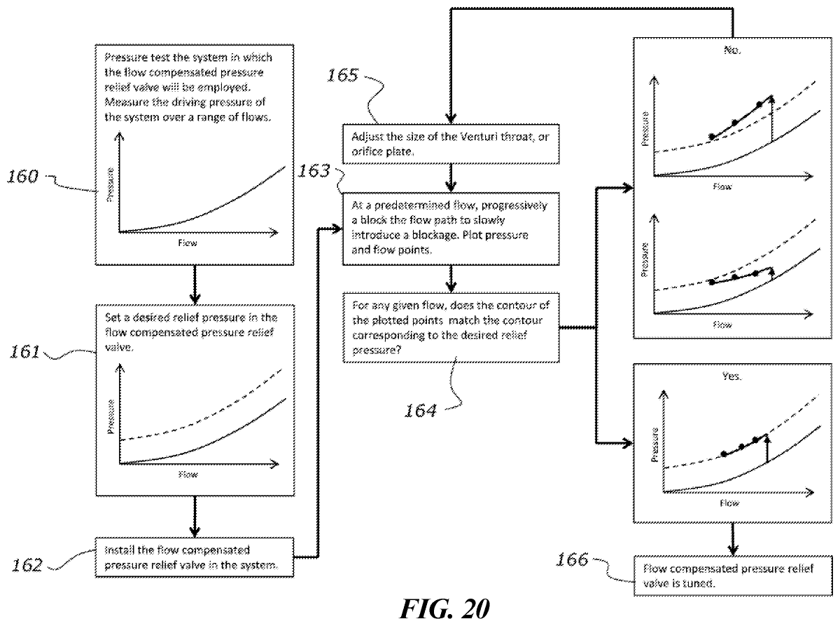

FIG. 20 illustrates a tuning method for tuning the FCPRV 100. At step 160 the system 10 is pressure tested to determine a system flow (e.g. flow delivered to the patient) versus the overall pressure drop response curve for the system 10. In step 161 a desired relief pressure v flow curve is determined, for example by adding an offset pressure to the system pressure v flow curve. At step 162, the FCPRV is installed in the system 10. At step 163, a flow restriction is then progressively added to the system downstream of the FCPRV 100, and the resulting relief pressure for a range of flow rates is determined to create a curve of measured pressure relief vs flow rate. At step 164, the actual pressure relief v flow curve is compared to the desired curve. At step 165, if the actual curve does not match the desired curve, the size of the flow restriction (Venturi throat or orifice) is adjusted and steps 163 and 164 are repeated again, until the desired pressure relief characteristic is achieved, at which point at step 166 the FCPRV 100 has been successfully tuned.

Alternatively or additionally the vent pressure threshold may be adjusted by adjusting any one or more of the other features of the PRV. For example, the tension in the valve membrane 105 may be adjusted by for example adjusting the relative position of the valve inlet 101 to the valve member 105, or the size of the vent outlet 103. In the PRV 100, the size of the vent outlets determine the shape of the pressure relief valve relief pressure v flow curve and therefore the vent pressure threshold over a range of flow rates. When the system is completely blocked/occluded, the FCPRV operates as the PRV described earlier, except that the sensing member may provide some additional bias to the valve member 105. Also, the biasing force provided to the valve member 105 by the sensing member 155 may be adjustable. For example the length of the mechanical link 157 between the sensing and valve members may be adjustable, a shorter length link increasing the biasing force and therefore the vent pressure.

FIGS. 2 and 3 show the FCPRV 100 with one embodiment of a connector 200 for coupling the FCPRV to a conduit for the supply of gas to a patient. . The embodiment of the connector 200 shown in FIG. 2 is a single part. The connector 200 is a male connector. The connector 200 is configured for use with a second connector, which is a female connector provided by the FCPRV. An example of a female connector is the valve body 110 at the outlet 205, as shown in FIGS. 1C and 2. Other examples of second connectors are described later in this specification.

Referring to FIGS. 2 and 3, features of one embodiment of the connector 200 will now be described. The connector 200 has a connector body with an inlet 203 and an outlet 205. The inlet 203 and outlet 205 define a gas flow passage therebetween. In some embodiments, the gas flow passage is or comprises a pressure line. The gas flow passage is defined at least in part by a wall 207 of the connector 200. The wall 207 provides a connector that is tubular component having a generally cylindrical shape that may be tapered and/or vary in its cross sectional area along the length of the connector 200. In other embodiments, the connector 200 comprises other cross-sectional shapes, e.g. elliptical, oval, obround, square and rectangle.

The connector body has an overlap portion 201 that is configured to overlap with a portion of the second connector, when connected. The connector 200 has an access passage, an access aperture, or an access hole, extending through the overlap portion 201 to the gas flow passage. The access passage fluidly communicates with the gas flow passage of the connector to enable sensing of the pressure in the gas flow passage. In this embodiment, the access passage comprises an aperture 211. In the embodiment shown in FIGS. 2 and 3, the aperture extends through the wall 207 of the connector 200. This embodiment has a single aperture 211. The aperture 211 has a similar size and shape to that of the bleed line 111. In alternative embodiments, there may be more than one aperture 211 extending through the wall 207. The connector 200 may have alignment features (not shown) to guide the connector towards the correct alignment position to ensure the aperture 211 is aligned with the bleed line 111. Examples of alignment features include two dimensional features such as text, symbols, and arrows. Other examples of alignment features include three dimensional features such as complementary protrusions and recesses. In various embodiments, the connector 200 may have one or more alignment positions with respect to the main outlet 153 of the FCPRV 100, to facilitate obtaining or not obtaining a flow and/or pressure compensated response from the valve 100 or not obtaining any pressure relief from valve 100. In a first configuration, the aperture 211 is not aligned with the bleed line 111 of the sensing mechanism and so there is no fluid communication between the gas flow passage through the connector 200 and the sensing chamber 154 via the access passage 211 such that the valve 100 will not provide any pressure relief functionality but will still allow gases to flow through the flow passage between main inlet 151 and main outlet 153. In such a configuration, the valve does not function as a pressure relief valve. In a second configuration, the aperture 211 is aligned with the bleed line 111 such that there is fluid communication between the gas flow passage through the connector 200 and the sensing chamber 154 via the bleed line 111 of the sensing mechanism and the access passage 211. The FCPRV 100 thereby functions as a flow and/or pressure compensated pressure relief valve as described above.

External features of the connector 200 preferably seal with internal features of the second connector, for example, the main outlet 153 of the valve body 110. In this embodiment, a portion of the exterior surface of the connector 200 is tapered. The surface is tapered inwardly towards the terminal end (inlet 203) of the connector 200. The taper is preferably a constant taper. The connector body tapers outwardly from the terminal end, from a smaller diameter to a larger diameter. In other embodiments, the connector 200 may have a constant diameter.

The main outlet 153 of the valve body 110 has a complementary size and taper such that the components preferably seal when assembled. Further embodiments are described below in which the connection between the main outlet 153 and the connector create the effect of a low pass filter between the flow passage through the connector and the sensing mechanism. In this embodiment, there is no low pass filter effect because a cavity is not formed between the walls of the main outlet 153 and the connector 200, where the cavity is in fluid communication with the gas flow passage and bleed line 111.

The connector 200 may comprise a stop. In the embodiment shown, the stop is a shoulder 209. The shoulder 209 is integral with the connector body. The shoulder 209 is positioned to abut the terminal end of the FCPRV outlet 153/second connector when the connector 200 is assembled with the FCPRV body, thereby to prevent, or at least substantially inhibit the connector 200 being over-inserted into the second connector.

The connector 200 may further comprise an engagement mechanism configured to couple the connector to the FCPRV 100. In the embodiment shown in FIGS. 2 and 3, the fit between the connector 200 and the main outlet 153 of the valve body 110 acts as an engagement mechanism. That is, the connector 200 is retained in place due to frictional forces between the internal walls of the second connector/main outlet 153 and the external surface of the connector 200.

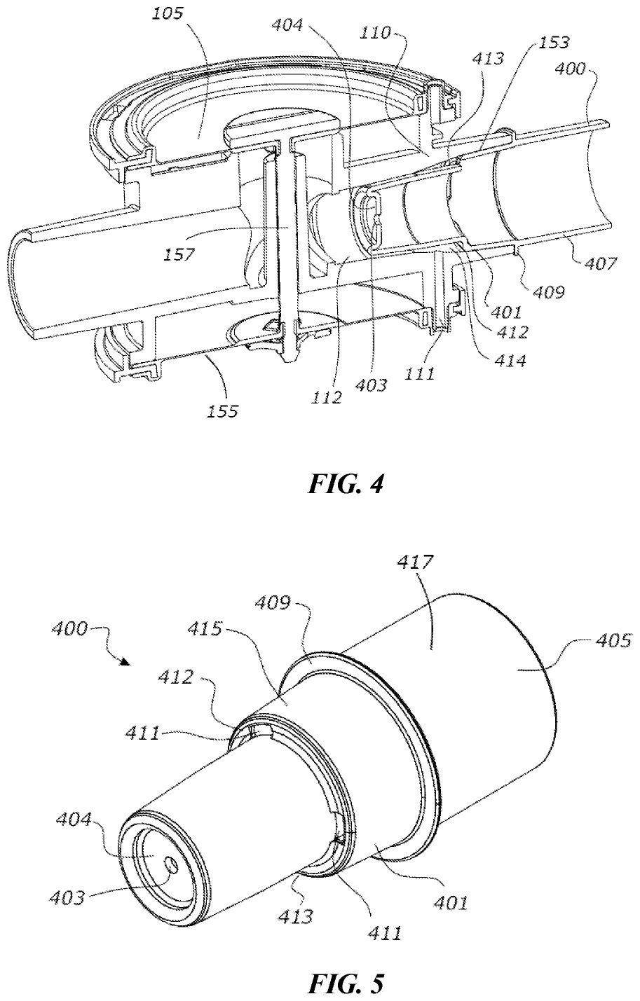

Another (second) embodiment of the connector will now be described with reference to FIGS. 4 and 5. The connector 400 has the same features and functionality of the first connector 200, unless described below. Like numbers are used to indicate like parts with the addition of 200.

In this embodiment, the connector has a cavity forming portion 413 and a sealing mechanism 415. When the connector 400 and the valve 100 are assembled, the sealing mechanism 415 substantially pneumatically seals the connector 400 and the main outlet 153 of the valve body 110. The cavity forming portion 413 and the main outlet 153 of the valve body 110 form a cavity.

The cavity forming portion 413 is a recess or change in a surface of the connector body that faces away from the gas flow passage. The exterior surface of the cavity forming portion has a shape that is not complementary to the internal surface of the main outlet 153 of the FCPRV 100, such that when assembled, the surfaces may be configured (e.g. having converging, diverging and/or parallel portions) to form a cavity 414. In the embodiment, a recess is provided by a stepped portion of the connector outer surface, while the main outlet 153 of the valve body 110 does not have a complementary shape. Rather, the main outlet 153 of the valve body 110 has a gradual taper such that when assembled, the connector 400 and main outlet 153 define a cavity 414 therebetween. In other configurations, the main outlet 153 of the valve body 110 may not have a taper. The cavity 414 is defined by an internal surface of the main outlet 153 of the valve body 110 and the cavity forming portion 413, when the connector 400 is coupled to the main outlet 153. In addition to having a stepped portion, the cavity forming portion 413 comprises an arcuate (includes but is not limited to curved) surface, preferably a radial surface. The arcuate surface is defined by the cylindrical connector body.

When formed, the cavity 414 is in fluid communication with the bleed line 111. The formed cavity 414 is in fluid communication with the gas flow passage via the access passage 411. The access passage comprises one or more apertures 411. This arrangement allows the communication of pressure in the gas flow passage through the apertures 411 into the cavity 414 and then subsequently into the bleed line 111 and the second chamber 154b, which can create a pressure differential across the sensing member 155 in the sensing chamber 154 so that the FCPRV 100 can function as described above.

In the embodiment shown, the cavity forming portion 413 has a longitudinal dimension along a longitudinal axis that is substantially parallel to a direction of gas flow in the gas flow passage. In an alternative embodiment, the cavity forming portion 413 may not be substantially parallel to a direction of gas flow in the gas flow passage. In this embodiment, the one or more apertures 411 are arranged substantially parallel or substantially perpendicular to a direction of gas flow in the gas flow passage. The location and formation of the cavity 414 in relation to the bleed line 111 or opening of the bleed line 111 can vary, provided it is in fluid communication with the bleed line 111 via the apertures 411.

In this embodiment, apertures 411 are arranged on the stepped portion/shoulder 412 formed between the cavity forming portion 413 and the sealing portion 415. This embodiment includes three apertures 411 that are radially arranged about the gas flow passage. There may be more apertures 411, for example, four or five apertures 411. There may be fewer apertures 411, for example, one or two apertures.

At least one aperture 411 may be in fluid communication with the gas flow passage via another aperture, the apertures being connected and in fluid communication by a channel (for example, a port in the wall of the connector that allows for downstream sampling).

FIGS. 4 and 5 show the inlet end (terminal end) of the connector includes a wall 404 having an inlet aperture 403 that provides a flow restriction or an additional flow restriction. The inlet aperture 403 is also the inlet of the connector 400. The wall 404 is spaced inwardly from the end of the connector, forming a recess. The wall 404 is located slightly inwardly, spaced from the terminal end, which increases the stiffness of the terminal end. The inlet aperture 403 is a tuning aperture in conjunction with a radial clearance, as described below. In an alternative embodiment, the wall 404 and inlet aperture 403 may be arranged directly at the terminal end of the connector 400. In another alternative embodiment, the aperture 403 may be absent, that is, the wall 404 is a continuous wall. In such an embodiment, all of the gas flows through the access passage apertures 411.

The sealing mechanism 415 is configured to form a first seal with a portion of the main outlet 153 of the valve body 110. The sealing mechanism may comprise one or more of sealing mechanisms known in the art, e.g. a face seal, an O-ring, a lip seal, a wiper seal, or a sealing surface. In the embodiment shown in FIGS. 4 and 5, the sealing mechanism is a sealing surface 415.

The cavity 414 is upstream of the sealing mechanism. In this case, the seal comprises an outer seal, that is, a seal that is proximate the terminal end of the main outlet 153 and/or proximate the collar 409 of the connector of FIG. 4, for the valve to function. FIG. 4 shows an example of an embodiment with this outer seal, as the sealing surface, where the outer seal is formed by engagement or interaction of a portion of the exterior wall of the connector 400 with a portion of the interior wall of the main outlet 153. It should be noted that other embodiments described with more than one seal could also be implemented with a single sealing surface. An outer seal can be defined as a seal that is downstream of the bleed line 111 and cavity 414 that is formed.

By providing a PRV body 110, and a separate connector 400, it is possible for the features of the PRV body to be set or fixed, while the pressure relief characteristics can be readily tuned by altering and/or adjusting the features of the connector or changing the connector used. Rather than providing a large number of different FCPRVs, it is possible to provide one design of a PRV body and a variety of different connectors. Each connector can be specifically tuned to provide the desired features, functionality and/or pressure relief characteristics, for example sealed and unsealed respiratory systems, and differentially sized patient interfaces (e.g. nasal cannulas). For example, at step 165 of the tuning process illustrated in FIG. 20, the size of the flow restriction can be adjusted by changing the connector to one having a different sized inlet 403.

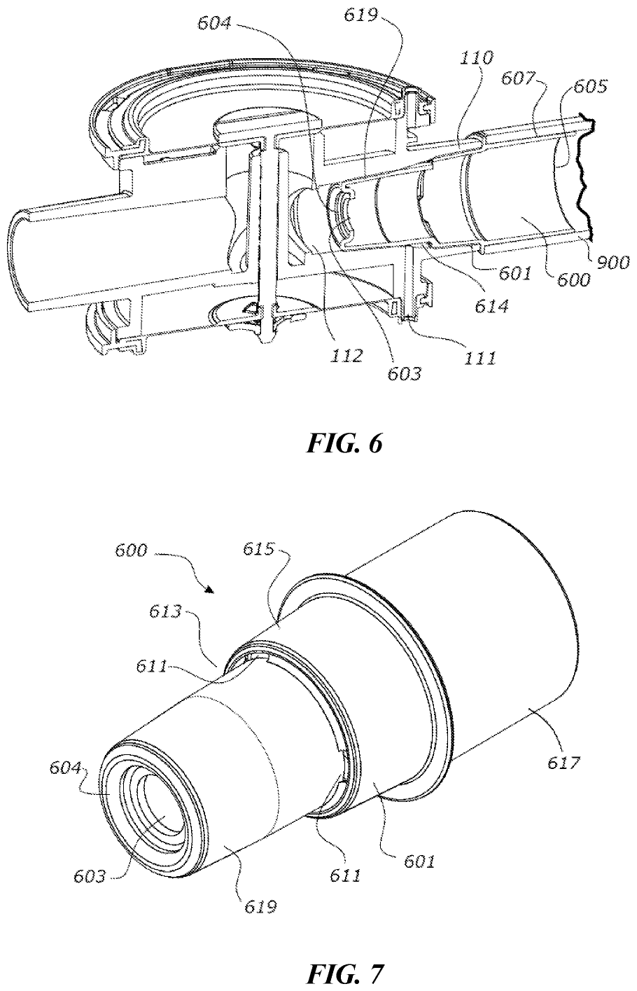

As shown in FIG. 6, in some embodiments an additional inner seal 619 may be present. An inner seal 619, described further below, comprises a seal that is upstream of the bleed line 111 and cavity 614 that is formed. This inner seal may be proximate to the center of the pressure relief valve when the connector 600 is engaged with the main outlet 153.

In alternative embodiments, other configurations of the connector and the valve body 110 may be used to form the cavity 414, 614. For example, the main outlet 153 of the valve body 110 may have a stepped portion and the connector may have a gradual taper. In another alternative embodiment, the main outlet 153 of the valve may have a taper and the connector may have a different taper. In another alternative embodiment, the main outlet 153 of the valve body 110 may have a stepped portion change and the connector 400 may have a stepped portion change, where the stepped portion changes are offset in a direction that is parallel to the direction of gas flow, forming a cavity. Further, the shape of the connector 400 and the shape of the main outlet 153 of the valve body 110 or other parts of the valve, together with the configuration of those components when assembled may be chosen or designed such that there is a tolerance and the components do not have to line up exactly to form a suitable cavity.

In the embodiment 400 of FIGS. 4 and 5, a radial clearance, at high fluid velocity, occurs. Flow accelerates through the apertures 411 and creates low pressure areas. In this embodiment there is an annular cavity 414 created that is sealed only at one end (outer seal). The size of the annular cavity 414 between the connector 400 and the internal wall of the main outlet 153 of the valve body 110 where there is no seal will have to be taken into account so that venting occurs as desired. As the cavity is only sealed at one end, the other end is in fluid communication with the gas flow passage, which may make tuning of the valve more difficult. The valve tuning has to take into account the leak flow into the cavity 414 which can affect the pressure differential across the sensing member 155 in the sensing chamber 154. Tuning the valve involves adjusting the size of the tuning aperture 403 or changing the diameter of the main outlet 153 and/or cavity forming portion 413 to change the size of the radial clearance to achieve a desired response. Changing the radial clearance will adjust the flow velocity. The relative sizes of the aperture 403 and the radial clearance will change the ratio of flow taking each path. This may be achieved by substituting a different connector with differently dimensioned inlet aperture 403, outlet 153, and/or cavity forming portion 413.

The connector may comprise a stop. In the embodiment shown, the stop is a collar 409. In the embodiment shown, the collar 409 is an annular collar. In alternative embodiments, the stop may be another feature that comprises the collar 409. The collar 409 is integral with the connector body. In an alternative embodiment, the collar 409 may be a separate component that is assembled with the connector body. A surface of the collar 409 may be configured to form a face seal with a surface of the second connector. In other configurations, the collar 409 may replace or aid the sealing mechanism 415. The collar 409 prevents, or at least substantially inhibits the connector 400 being over-inserted into the second connector.

Another (third) embodiment of the connector will now be described with reference to FIGS. 6 and 7. The connector 600 has the same features and functionality of the second connector 400, unless described below. Like numbers are used to indicate like parts with the addition of 200.

In this embodiment, there is a first sealing mechanism 615 and a second sealing mechanism 619. Embodiments of the connector having two sealing mechanisms facilitate tuning of the response of the FCPRV. The cavity forming portion 613 is between the first sealing mechanism 615 and the second sealing mechanism 619. The access passage is in fluid communication with the cavity 614. The access passage is also positioned between the first sealing mechanism 613 and the second sealing mechanism 615. In the embodiment of FIGS. 6 and 7, the cavity 614 that is formed between the first sealing mechanism 615 and the second sealing mechanism 619, when the connector 600 is coupled to the main outlet 153, is an annular cavity. That is because the main outlet 153 of the valve body 110 has a radial bore and the connector 600 has a radial outer surface.

In the embodiment shown in FIGS. 6 and 7, the second sealing mechanism 619 is a sealing surface. The first sealing mechanism 615 and the second sealing mechanism 619 are formed by the interference/friction fit of the external surfaces of the connector 600 and the complementary inner surface(s) of the main outlet 153 of the valve body 110 as shown. However, many other methods may be used to create seals and form a cavity. For example, O-rings, wiper seals, adhesives, foams or lip seals may be used at different locations on a connector and seal with internal or external surfaces of the female connector (valve body 110) to form the cavity 614. Further, an internal interference fit may be used for one seal in conjunction with retention features such as a tab and clip or other external sealing method on the outside of the valve/connection assembly to create a cavity.

FIG. 15 shows a simplified schematic cross section of a connection assembly where two O-ring seals of different sizes are used as an alternative sealing mechanism to form the cavity 1114. FIG. 15 shows the first sealing mechanism in the form of a relatively small O-ring 1115. FIG. 15 also shows the second sealing mechanism in the form of a relatively large O-ring 1119. The cavity 1114 is formed between the O-rings 1115, 1119. Depending on the size and shape of the connector and the body, the O-rings 1115, 1119 may be closer in size, the same size, or the O-ring of the first sealing mechanism 1115 may be larger than the O-ring of the second sealing mechanism 1119. The embodiment in FIG. 15 shows an opening, access aperture, or access passage 1111 in the overlap portion that communicates with the pressure line 111 via the cavity 1114.

Returning to FIGS. 6 to 8, which show a preferred connection assembly, in the embodiment shown, the overlap portion 601 includes the first sealing mechanism 615. The overlap portion 601 also comprises the second sealing mechanism 619. In an alternative embodiment, the overlap portion 601 may comprise only one of the sealing mechanisms.

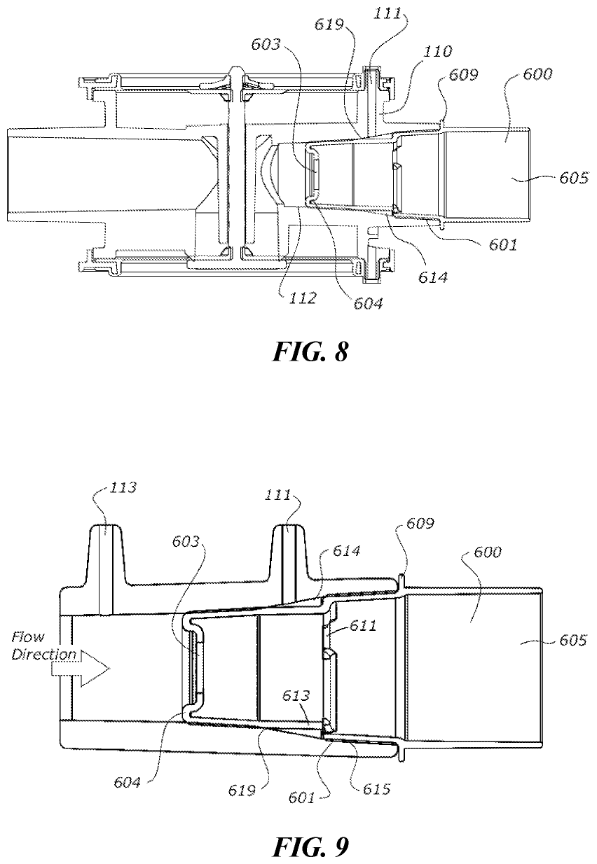

FIG. 8 shows the main outlet 153 of the valve body 110 has an internal, gradually tapered bore. This internal bore of the main outlet 153 of the valve body 110 has non-standard diameters. This is to avoid the connection of incorrect connectors with the main outlet 153. In this embodiment, the flow restriction is provided by the aperture 603 at the inlet to the connector (rather than by the valve body). If an incorrect connector is made that fits into the main outlet 153, the valve is unlikely to operate as a flow and/or pressure compensated valve or a valve that provides pressure relief because the valve and connector would not have a flow restriction and/or an access passage with the main gas flow path to achieve the flow rate and/or pressure sensing as described in relation to the embodiments of the valve and connector. In this case, if an incorrect connector that does not have a flow restriction but which provides fluid communication between the second sensing chamber and the main gas flow passage between main inlet 151 and main outlet 153, (e.g. via the communication line 111), is used with the FCPRV body, the pressure response of the valve 100 will match the response observed when the outlet 153 of the valve 153 is blocked and gases are venting from the valve. That could include a substantially flat response of, for example, 20 cm H₂O. If an incorrect connector that does not provide fluid communication between the second sensing chamber and the main gas flow passage between main inlet 151 and main outlet 153 (i.e. the communication line 111 is blocked), is used with the FCPRV body, the valve 100 will not provide any pressure relief during use but gases are still able to flow through the main flow passage. As a result, the respiratory system may not be able to deliver all of the prescribed flow rates to the patient or the flow is limited.

It is preferred that the connector and the main outlet 153 of the valve body 110 are pneumatically sealed such that there is not a significant leak of gas to atmosphere. In some embodiments, if there is a known or expected leak, the flow restriction may be adjusted (for example by altering the size of the tuning orifice) based on this known or expected leak such that expected valve function is maintained.

FIGS. 9 to 11 show some alternative connection assemblies. In FIG. 9, pressure sensing is provided by upstream and downstream pressure lines 113, 111. The downstream (first) pressure line 111 and the upstream (second) pressure line 113 are each coupled to a pressure sensing mechanism, e.g. a sensing membrane of a flow and/or pressure compensated pressure relief valve, a differential pressure sensor or multiple absolute or gauge pressure sensors. The pressure sensing mechanism can be sensing mechanism 150 where the first pressure line 111 is in fluid communication with the second chamber 154b and the second pressure line 113 is in fluid communication with the first chamber 154a. In various embodiments, the pressure sensing mechanism may be one that simply samples pressure upstream and downstream of the flow restriction defined by the aperture 603.

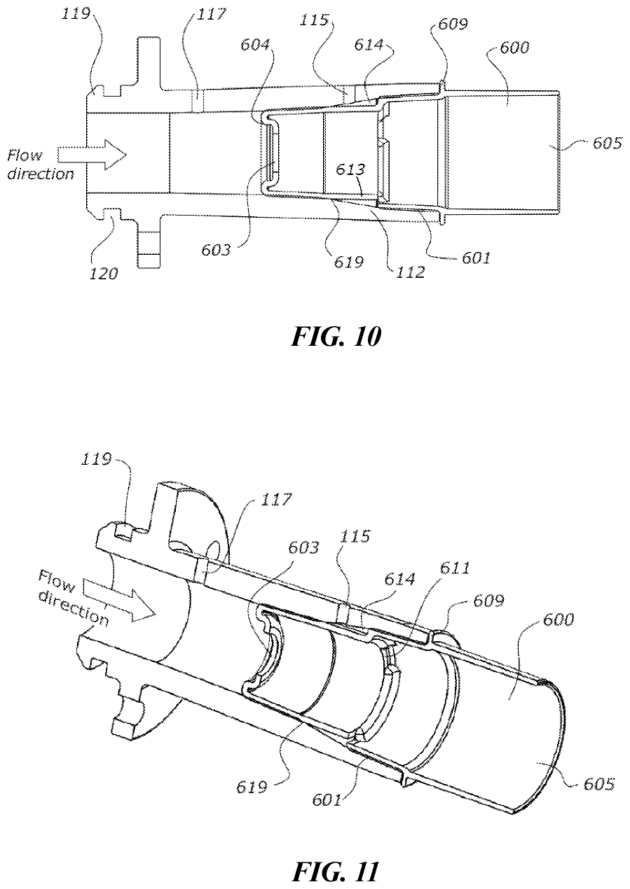

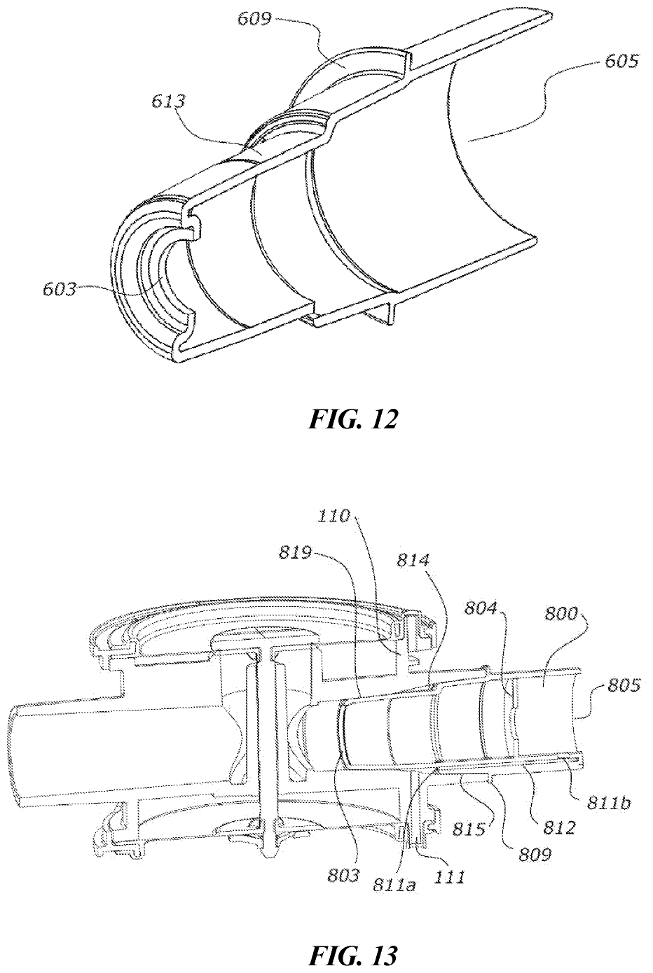

FIGS. 10 and 11 shows another alternative assembly in which the second connector is not formed by the outlet of a FCPRV, and instead is a connector for attaching the connection assembly to another circuit component, such as a manifold. Upstream and downstream pressure lines 117, 115 are provided through the wall of the second connector. The second connector may comprise an engagement mechanism configured to engage the second connector with the circuit component. FIG. 10 shows an engagement mechanism comprising a groove 120. The groove may receive a seal, such as an O-ring. The end 119 of the second connector can be received by the circuit component such that the o-ring seals against an interior surface of the circuit component. Alternatively, groove 120 may act as a snap fit type engagement mechanism whereby protrusions provided in the circuit component snap fit into the groove 120.

This engagement mechanism may also be in other common forms such as interference fit, twist/screw attachment, or a snap-fit. With reference to the embodiments in FIGS. 9 and 10, the cavity forming portion 613 is tapered relative to a direction of gas flow. In addition, FIGS. 9 and 10 show the connector tapering from a terminal end, from a larger diameter to a smaller diameter.

FIG. 13 shows a further alternative embodiment of the connector 800 that is used to sample pressure downstream. The connector 800 has the same features and functionality of the third embodiment connector 600, unless described below. Like numbers are used to indicate like parts with the addition of 200.

The embodiment in FIG. 13 shows a connector 800 with an opening or aperture 811a in the overlap portion 801 that communicates with another aperture 811b in the wall of the connector 800 that accesses the main gas flow path, via a pressure passage or line 812 within the wall of the connector 800. The pressure passage or line 812 connects the cavity 814 at the overlap portion 801 to the main gas flow path at a portion of the connector (or another circuit component), downstream of the main outlet 153. Compared to the earlier described embodiments, the pressure sampling aperture is a pressure sampling line defined by apertures 811a, 811b and pressure passage 812, where the aperture 811b is provided further downstream. The location of the pressure sampling aperture 811b (which could be located beyond the terminal end of the main outlet 153) and the pressure line 812 allows the flow restriction formed by the wall 804 and aperture 803 to be moved further downstream if desired, as shown in FIG. 13. If the flow restriction and/or location of pressure sampling 811 is desired to be moved, the pressure is sampled downstream of the flow restriction. The embodiment in FIG. 13 shows a pressure sampling line that may sample somewhere even further downstream. This provides more flexibility in the location of the flow restriction.

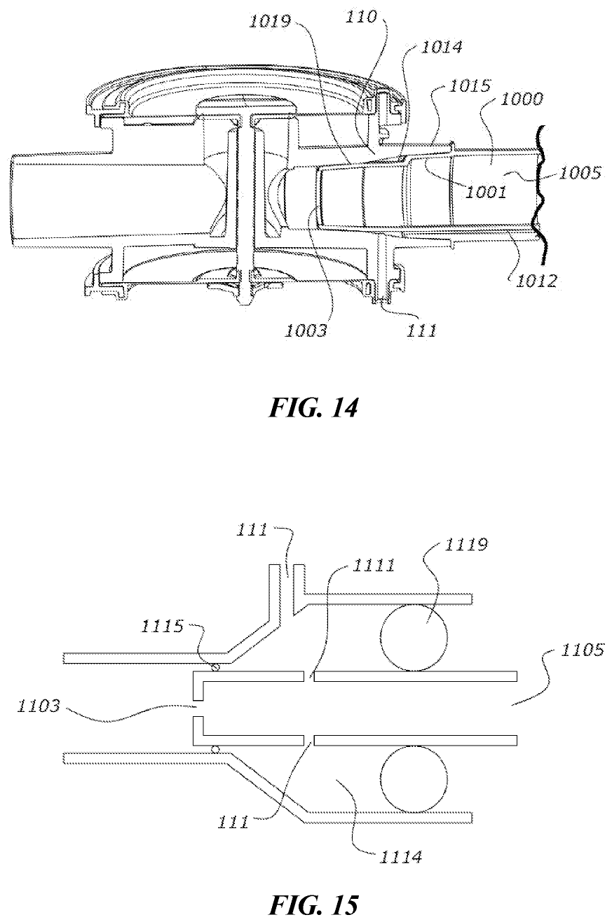

FIG. 14 shows a connector 1000 having regions with different diameters. The connector 1000 has the same features and functionality of the fourth embodiment connector 800, unless described below. Like numbers are used to indicate like parts with the addition of 200.

The embodiment in FIG. 14 shows a connector 1000 with a pressure passage or line 1012 within the wall of the connector 1000. The pressure passage or line 1012 may be substantially rigid, or may comprise a flexible conduit. The pressure passage or line 1012 fluidly connects the cavity 1014 at the overlap portion 1001 to the main gas flow path at a portion of the connector (or another circuit component), downstream of the main outlet 153. Similar to the fourth embodiment, the pressure sampling aperture is a pressure sampling line defined, in part, by pressure passage 1012. An aperture (similar to aperture 811b) is provided further downstream, and is not shown because it is further downstream than the features shown in FIG. 14. The aperture is much further downstream than the aperture of the fourth embodiment. The location of the pressure sampling aperture and the pressure line 1012 allows the flow restriction formed by the wall and aperture to be moved further downstream if desired. Again, they are not shown in FIG. 14 because they are further downstream than the features shown in FIG. 14. Similar to the fourth embodiment, the flow rate and/or pressure of the gases flow through the gas flow passage may be sampled downstream of the flow restriction 1003. By providing a pressure sampling line that may sample further downstream than the earlier embodiments, the connector provides more flexibility in the location of the flow restriction.

The region closest to the inlet 1003 has a diameter that is smaller than the region that is closest to the outlet 1005. The difference in the diameters causes the aforementioned interaction with the internal taper of the FCPRV body that forms the cavity 1014.

The apertures (not visible) vent flow into the cavity 1014 and subsequently into the diaphragm chamber. The apertures are located tangential to the vertical surface where the step change in diameter occurs. That is, these apertures are located adjacent to the direction of flow. Additionally or alternatively, the apertures may be located outwardly or at other locations on the connector provided they allow venting into the formed cavity 1014. Similar to the previous embodiment, the connector has a pressure line 1012 within the wall of the connector 1000.

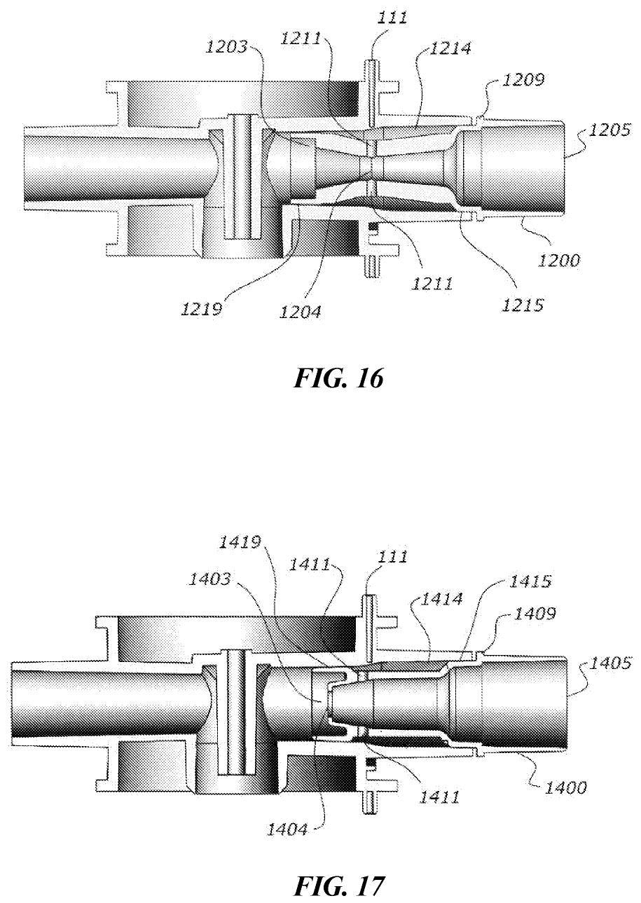

FIG. 16 shows another alternative embodiment of the connector 1200 that may be used. The connector 1200 has the same features and functionality of the third embodiment connector 600, unless described below. Like numbers are used to indicate like parts with the addition of 600.

The connector shown in FIG. 16 provides a cavity 1214 that is in fluid communication with the bleed line 111. The connector has one or more apertures 1211 that are in fluid communication with the gas flow passage. The embodiment shown in FIG. 16 has a venturi shaped connector profile that provides a flow constriction 1204. The aperture 1211 and the flow restriction 1204 are substantially aligned, hence the pressure is sampled at a point of high flow velocity. The annular cavity 1214 acts as a low pass filter. The low pass filter provides a damping effect. This low pass filter reduces turbulence in the flow and increases stability of the flow because the volume of the chamber is pressurised before the volume of the diaphragm is pressurised. The size of the cavity formed would affect this low pass filter but the orientation of the apertures does not.

FIG. 17 shows another alternative embodiment of the connector 1400 that may be used. The connector 1400 has the same features and functionality of the third embodiment connector 600, unless described below. Like numbers are used to indicate like parts with the addition of 800 respectively.

The connector shown in FIG. 17 provides cavity 1414 that is in fluid communication with the bleed line 111. The connector has one or more apertures 1411 that is in fluid communication with the gas flow passage. The exterior of the embodiment shown in FIG. 17 has multiple stepped portions, or step changes in diameter. Similar to the connector of FIG. 16, the annular cavity 1414 acts as a low pass filter.

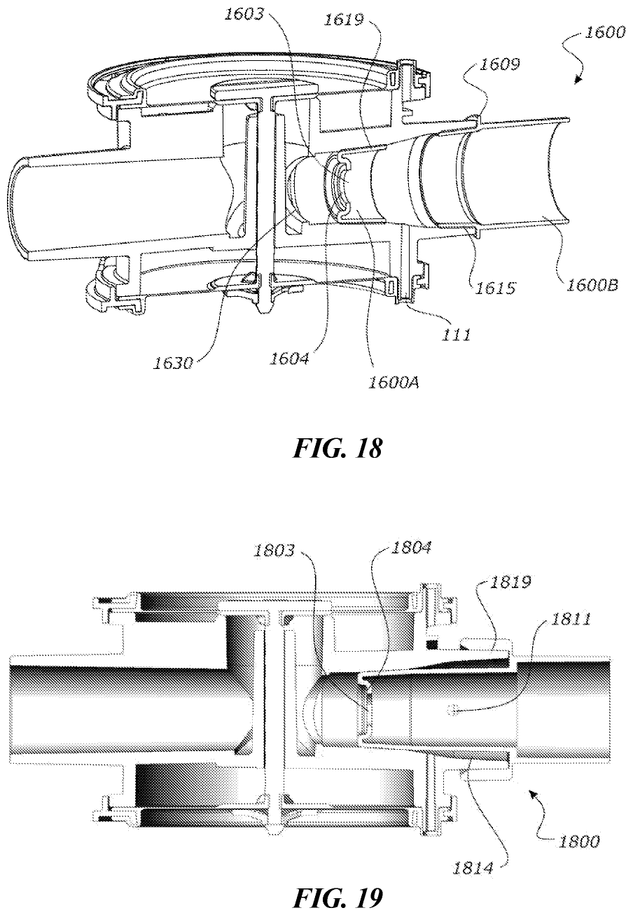

FIG. 18 shows an alternative form of the connection assembly where the connector 1600 comprises at least two parts, a first part 1600A and a second part 1600B. The connector 1600 has the same features and functionality of the third embodiment connector 600, unless described below. Like numbers are used to indicate like parts with the addition of 1000. The two parts 1600A and 1600B are separated by a gap. In this embodiment, the first part comprises a wall 1604 and aperture 1603 that provide a flow restriction. The interior of both parts 1600A and 1600B are in fluid communication with the bleed line 111. The first part 1600A, with the flow restriction 1604, also has a sealing mechanism 1619 that has the same features and functionality as the second sealing mechanism of the third embodiment connector 600. The second part 1600B also has a sealing mechanism 1615 that has the same features and functionality as the first sealing mechanism of the third embodiment connector 600. Optionally, these two parts 1600A and 1600B may be connected by a cable or tether or other mechanism so that both parts can be readily removed if desired. In some embodiments, the first part 1600A may be permanently connected, or configured to be permanently connected to the valve body 110 or the main outlet 153, for example as a multiple use part that is re-used multiple times. The second part 1600B may be removable, for example as a single-use part. The first part 1600A, may have any form that produces a pressure drop. For example, the first part 1600A may be or comprise a permeable material (such as foam, porex, membrane, etc), with or without an aperture, or an insert with one or more apertures.

In some applications, the valve 100 itself may produce a portion of the pressure drop, with the connector 1600 providing an additional pressure drop portion. In addition to a small pressure drop as fluid flows through the valve 100 from the main inlet, there may be two main areas each with a substantially stepwise pressure drop when the connector is connected. An example of a feature that produces a pressure drop is the relatively large orifice 1630 shown in FIG. 18. Other forms or variations of the valve having an aperture within the valve that produces a pressure drop are show in FIGS. 2, 4, 6, 8, 13, 14, 18, and 19, for example. Preferably a majority of the pressure drop through the valve and connector assembly is provided by the aperture 403, 603, etc. provided by the connector.

Another embodiment of the connector will now be described with reference to FIG. 19. The connector 1800 has the same features and functionality of the third connector 600, unless described below. Like numbers are used to indicate like parts with the addition of 1200.

In this embodiment, the second sealing mechanism 1819 is a female seal compared to the male seal of the third embodiment of the connector 600. That is, the seal is provided by a wall or surface of the connector 1800 that faces towards the gas flow passage compared to the third embodiment of the connecter 600 in which the sealing surface faces in a direction away from the gas flow passage.

In the embodiment shown in FIG. 19, the second sealing mechanism 1819 is a sealing surface. The second sealing mechanism 1819 is formed by the interference/friction fit of an internal surface of the connector 1800 and the complementary outer surface(s) of the main outlet 153 of the valve body 110 as shown. However, many other methods may be used to create the secondary sealing mechanism and form the cavity 1814. For example, O-rings, wiper seals, adhesives, foams or lip seals may be used.

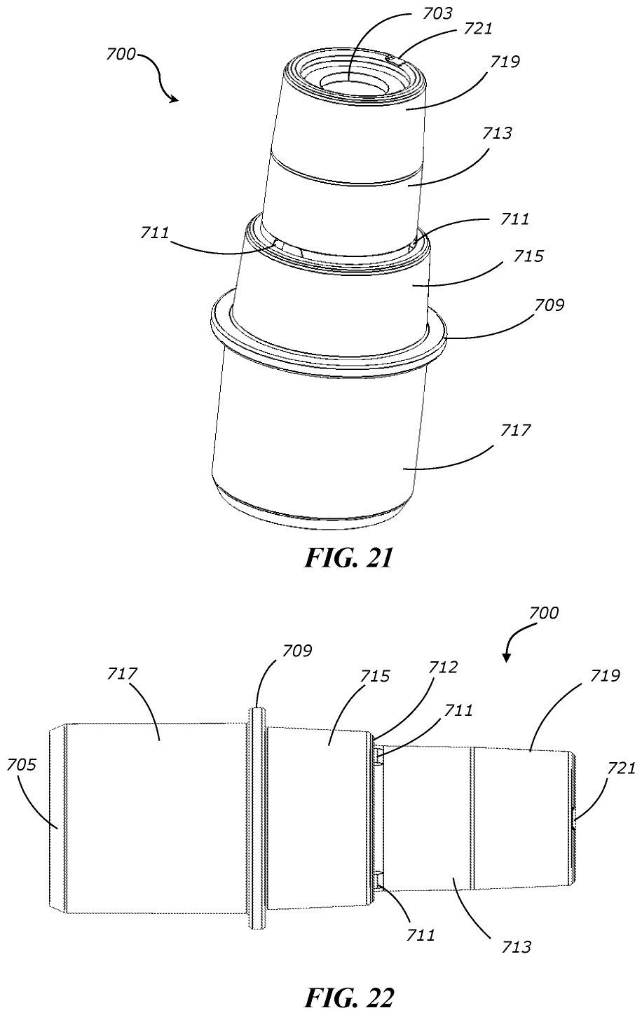

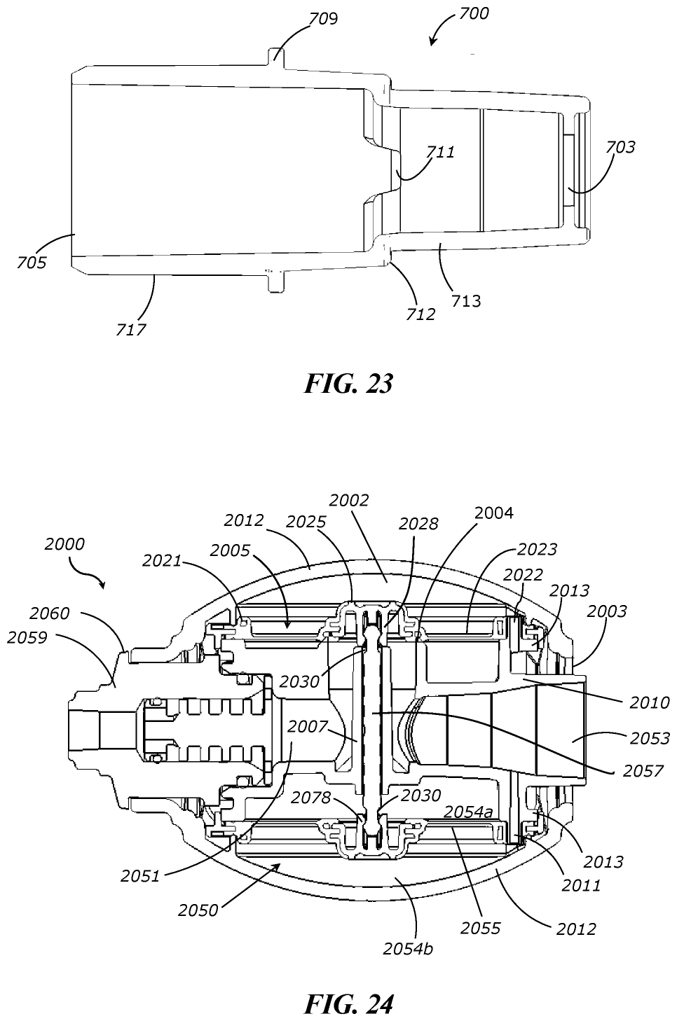

Another embodiment of the connector will now be described with reference to FIG. 21. The connector 700 has similar features and functionality to the third embodiment connector 600, unless described below. Like numbers are used to indicate like parts with the addition of 100.

In the connector 700 of FIG. 21, the apertures 711 in the wall of the connector for fluid communication with the FCPRV sensing mechanism 150, are provided in the cavity forming portion of the connector 713. The apertures 711 are positioned adjacent to the shoulder 712 that is formed between the cavity forming portion 713 and the overlap portion 715. Fluid flow through the apertures 711 is substantially perpendicular to the main flow direction through the connector 700 from the inlet aperture 703 to the outlet.

The apertures 1211, 1411, 711 provided in the wall of the connector 1200, 1400, 700 for sensing pressure are preferably provided in a region of laminar or low-turbulence flow. For example, the apertures may be provided in the wall of the connector at, or immediately adjacent and downstream of, the flow restriction.

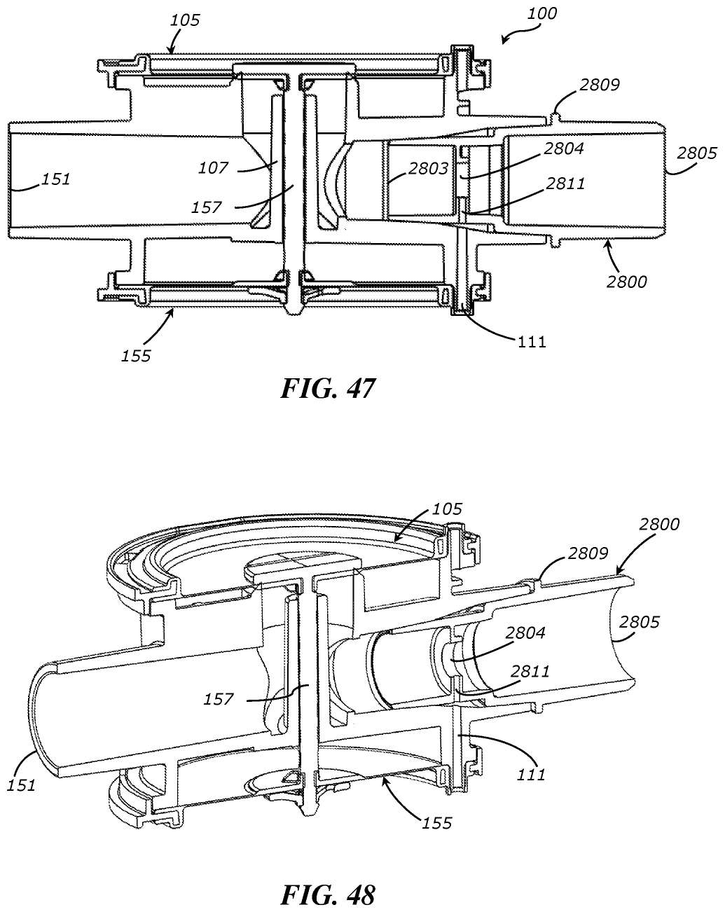

FIG. 16 shows and exemplary connector 1200 having a venturi-type flow restriction 1204, with the sensing apertures 1211 provided at the narrowest point of the venturi. FIGS. 47 and 48 show an alternative embodiment connector 2800 (shown assembled with the FCPRV 100 described above) with the sensing aperture 2811 also provided at the flow restriction 2804. In this embodiment, the flow restriction 2804 is an orifice plate type restriction, with the one or more sampling apertures 2811 provided by one or more channels in the wall of the orifice plate, in fluid communication with the orifice 2804.

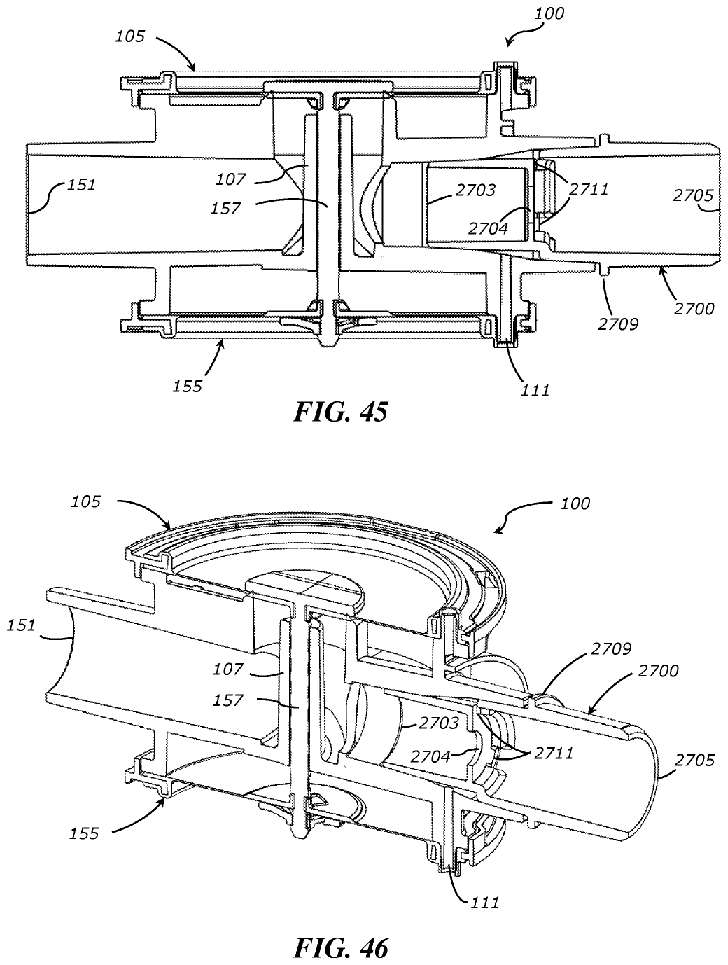

Alternatively, the one or more sampling apertures may be provided immediately adjacent the flow restriction, preferably on a downstream side of the flow restriction. FIGS. 45 and 46 illustrate an alternative embodiment connector 2700 in which the sampling apertures 2711 are provided in a shoulder of the connector 2700, immediately downstream of and adjacent the flow restriction 2704. Providing the sampling apertures as close as possible to the flow restriction is advantageous because the gas flow at these points is more likely to be laminar or have low turbulence compared with flow at points further downstream of the flow restriction.

The connectors 2700 and 2800 have similar features and functionality to the third embodiment connector 600, unless otherwise described. Like numbers are used to indicate like parts with the addition of 1100 or 1200 respectively.

A moulding notch 721 may be present at the upstream, inlet end of the connector 700, for ease of manufacturing of the connector, for example by injection moulding.

In some embodiments described herein, the connector is provided as a male member and the outlet port of the valve is provided as a female member, where the connector is received by the outlet port to form the gas flow passage. In other embodiments and/or configurations, the connector may be provided as a female member and the outlet port of the valve may be provided as a male member, where the outlet port is received by the connector to form the gas flow passage. Also in other embodiments and/or configurations, the connector may comprise male and/or female parts that correspond to, engage and/or couple with complementary female/male parts of the outlet port of the valve, for example as shown in FIG. 19.

In some embodiments described herein, the cavity forming portion may be tapered relative to a direction of gas flow. An example is when the gas flow passage is or comprises a pressure line. The connector may taper towards a terminal end, from a larger diameter to a smaller diameter.

In some embodiments, the connector may be configured to be coupled to a pressure relief valve. In particular, the connector may further comprise an engagement mechanism configured to couple the connector to a pressure relief valve. Suitable engagement mechanisms include clips, complementary threaded portions, or press fits. In the embodiments shown, the engagement mechanism is a press fit.

In some embodiments, the pressure relief valve may be a flow and/or pressure compensated pressure relief valve. In some embodiments, the pressure relief valve may be a flow compensated pressure relief valve or a pressure compensated pressure relief valve. The pressure line may be in fluid communication with a sensing chamber of the pressure relief valve. The pressure relief valve may comprises a sensing member configured to sense a pressure differential between the sensing chamber and a main gas flow passage that provide gas flow to a patient. Movement of the sensing member changes the venting pressure of a valve member.

In some embodiments, the pressure line is a first pressure line and the connector further comprises a second pressure line that is upstream of the first pressure line. The first pressure line and the second pressure line may each be coupled to a pressure sensing mechanism.

In some embodiments, the connector may be configured to be coupled to a respiratory circuit component. For example, the connector may comprise an engagement mechanism configured to engage the connector with the respiratory circuit component. Suitable engagement mechanisms include clips, complementary threaded portions, or press fits.

Some of the described embodiments indicate a direction of flow. However, in all described assembly embodiments that the direction of gas flow can be either direction. The terms ‘upstream’ and ‘downstream’ used herein, are dependent on the direction of flow in for example the gas flow passage.

Any one of the connectors described herein may be releasably or permanently secured to, or integral with, the end of a conduit. An example of a conduit 900 is shown in FIG. 6. The connector may be assembled with the conduit during manufacturing, or after manufacturing. The conduit may be any suitable conduit. The conduit will be chosen or designed depending on a variety of factors. Those factors include the location of the pressure relief valve in the circuit, and/or the location at which pressure sensing is desired.

The connector may be configured to releasably attach to the end of an existing conduit to enable the existing conduit to be used with the pressure relief device described herein. The connection between the conduit 900 and the connector 400 may be by way of an interference fit, for example, where the conduit connecting portion 417 of the connector is received by the conduit 900 and seals against the internal wall surface of the conduit. Alternatively, connecting portion 405 of the connector may receive the conduit and form an interference fit with the outer surface of the conduit.

This conduit with the connector 100 is then connected to the PRV body, forming a connection assembly. In a preferred embodiment, the connector is attached to the end of a conduit during manufacturing. The connector and the conduit are then connected to the PRV by a user. The conduit may be part of a circuit between a flow source and a humidifier or between a pressure relief valve and a humidifier. For example, the conduit may extend from a flow source to a humidifier. The conduit may be referred to as a dry line when it connects an outlet of a flow source or a pressure relief valve to an inlet of a humidifier or humidification chamber, and the gases that it transports is not humidified. Furthermore, additional components may be included to modify the circuit (e.g. a gas flow modulator) and the dry line may extend from a flow source to one of these additional components or from the additional components to a humidifier or humidification chamber. In some embodiments, a gas flow modulator receives a gases flow from a flow source and the connector and conduit are connected to an outlet of the gas flow modulator to deliver the gases flow from the gas flow modulator to a humidifier or humidification chamber for the gases flow to be humidified. A gas flow modulator may be a gas flow modulator having features described in WO/2017/187390, the entirety of which is hereby incorporated by reference herein.

The interaction between the connector that is integral with or coupled to the dry line and the valve is an interference/friction fit in the preferred embodiment. However, other methods may be employed such as a twist/screw attachment or an external engagement mechanism, e.g. adhesives (includes but not limited to glues, chemical bonding, etc), overmoulds and welds.

Each of the connectors described herein allows alterations or modifications to the tuning orifice to be readily made by changing the connector rather than the entire valve. Further, the connectors described discourage connection of incorrect connectors to the valve because the valve will not function as desired unless the connector is a connector having the features and functionality of one of the embodiments described here, or unless the connector is tuned appropriately (e.g. size of the flow restriction) for the resistance to desired flow of the circuit and patient interface.

FIG. 24 shows a further embodiment FCPRV 2000. The FCPRV 2000 has similar features and functionality to the FCPRV 100 of FIGS. 1C and 2, unless described below. Like numbers are used to indicate like parts with the addition of 1900.

The FCPRV 2000 comprises a device inlet 2051 and a device outlet 2053, with a main gas flow passage between the inlet and the outlet. A pressure relief mechanism is connected between the inlet and the outlet and comprises a valve seat 2004 and a valve member in the form of a diaphragm component 2005, described in detail below. In some embodiments, the diaphragm component 2005 is a removable diaphragm component. A portion of the diaphragm component 2005, for example, the diaphragm and/or the link connector portion, is arranged to be seated against the valve seat 2004 in a first configuration and to be spaced from the valve seat in a second configuration when pressure in the gas flow passage exceeds a pressure threshold, to provide pressure relief. In some embodiments, the base of the link connector portion may comprise a layer of the overmoulded diaphragm, and this overmoulded portion may be seated against the valve seat in the first configuration.

The FCPRV 2000 comprises a sensing mechanism 2050 to dynamically adjust the pressure threshold based on a flow rate of a flow of gases at or through the outlet 2053. The sensing mechanism comprises a sensing member in the form of a diaphragm component 2055, for permanent or releasable attachment to a valve adjustment member 2057. In some embodiments, the diaphragm component 2055 is a removable diaphragm component. In some embodiments, the diaphragm component 2055 is releasably attachable to the valve adjustment member 2057. The valve adjustment member 2057 operatively couples the valve member and the sensing member of the pressure relief mechanism to change the relief pressure of the pressure relief mechanism.

The embodiment shown in FIG. 24 comprises a coupler 2059 at a main inlet portion (which houses main inlet 2051), for coupling a flow source to the device inlet 2051. The coupler comprises a flange or lip 2060 that extends over the edge of the main inlet portion to prevent fluid or debris entering the inlet 2051. In some embodiments, the coupler comprises engagement features for engaging the inlet 2051 and/or chamber caps 2012. In some embodiments the coupler comprises sealing features (e.g. o-rings) for sealingly engaging with the inlet 2051 and/or chamber caps 2012. In some embodiments, the coupler 2059 couples with the inlet 2051 via an interference fit.

In the embodiment shown the coupler 2059 comprises a muffler. Additionally or alternatively, in some embodiments, the coupler may provide an adaptor for connecting to different flow sources.

The chamber caps 2012 define an aperture 2003 adjacent the device outlet 2053, through which air at atmospheric pressure can enter the valve chamber 2002, and through which gases released by the pressure relief mechanism can escape. The aperture 2003 may comprise a filter (not shown) to prevent the ingress of dust and contaminants into the device 2000 and to reduce noise emitted by the valve during venting. The filter comprises a porous, air permeable material.

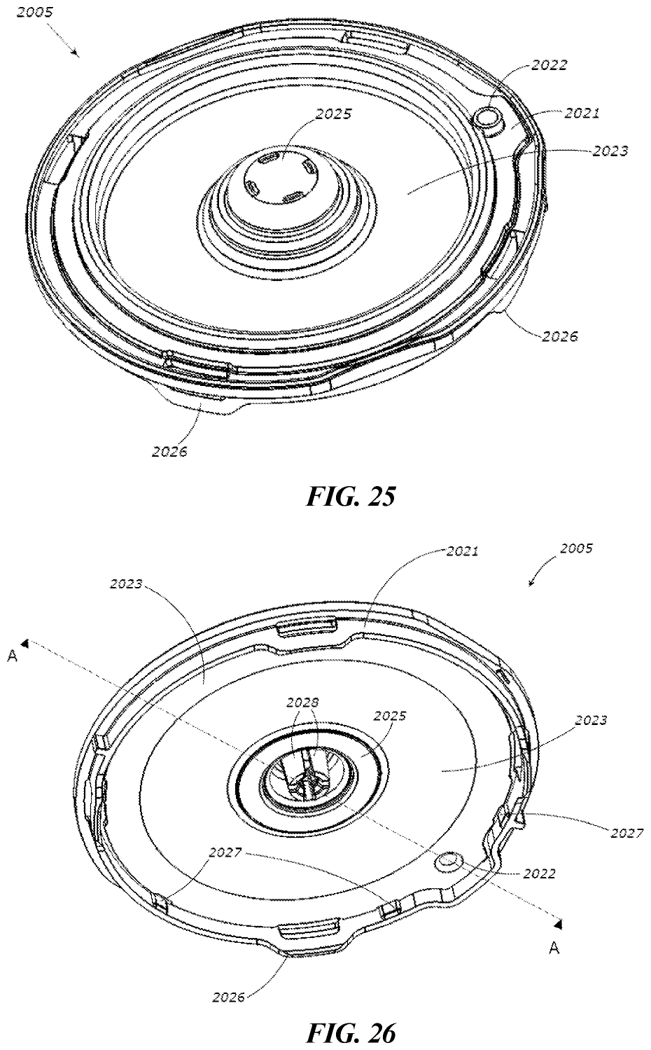

In the embodiment of FIG. 24, the valve member and the sensing member are each provided by diaphragm components 2005, 2055 illustrated in FIGS. 25 to 29B. In this embodiment, the diaphragm component 2005 comprising the valve member is identical to the diaphragm component 2055 comprising the sensing member. However, in other embodiment FCPRVs, the diaphragm components 2005/2055 for the valve and sensing members may differ.

The diaphragm component 2005/2055 comprises a flexible diaphragm 2023/2073 and a substantially rigid link connector portion 2025/2075. A portion of the diaphragm 2023/2073 is overmoulded to the link connector portion 2025/2075 to join the diaphragm to the link connector portion. The rigid link connector portion 2025/2075 is configured to attach to a valve adjustment member such as the mechanical link 2057.

The diaphragm component 2005/2055 further comprises a frame 2021/2071. The frame 2021/2071 is an annular and substantially rigid frame, although other shapes of the frame are possible. The substantially rigid frame 2021/2071 may be formed from any suitably rigid material, such as a metal, plastic, or composite material, for example, glass-filled polybutylene terephthalate (PBT), glass-filled nylon, polycarbonate or other plastics material known in the art. Each frame 2021/2071 is seated and seals against complementary rims provided on the FCPRV body 2010. The frame may comprise one or both of engagement features and location features for attaching the frame to the FCPRV body 2010 and/or to the chamber caps 2012. The engagement features enable attachment of the frame 2021 and thereby the diaphragm component 2005 to the body 2010 of the FCPRV 2000.

Referring to FIGS. 25 to 27, which show the valve member diaphragm component 2005, the engagement features comprise a plurality of clips 2026 to engage corresponding engagement features on the body of the FCPRV. In the embodiment shown, each clip 2026 comprises an aperture or recess to receive a detent, catch, or protrusion provided on the body of the FCPRV. The clips 2026 protrude in a first direction from the frame 2021 and comprise a rectangular recess or aperture, but other shapes of clips and apertures are envisaged. The clips 2026 may have some flex such that they can flex and engage with the engagement features on the body, or the clips 2026 may be substantially rigid with the frame. The engagement of the clips 2026 of the frame 2021 to the valve body 2010 may produce an audible or haptic feedback to indicate that the engagement is complete. The engagement features on the body of the FCPRV may be provided by protrusions extending outwardly from the rim 2013 on which the frame is seated.

The frame 2021 in the embodiment shown comprises four engagement clips 2026 spaced equally around the perimeter of the frame. However, alternative embodiments may comprise more or fewer engagement features.

The frame 2021 of FIGS. 25 to 27 further comprises location features to assist with correctly orienting the the diaphragm component 2005 when it is held or clipped to the valve body 2010. In the embodiment shown, the location features 2027 comprise a plurality of projections projecting radially inwardly from a surface of the frame. The location projections abut an interior wall of the rim 2013 to help to ensure the diaphragm frame 2021 is concentric with the opening in the valve body 2010, by reducing any play between the two components.

In some embodiments, the body 2010 of the FCPRV may comprise complementary recesses to receive the location projections 2027. In such an embodiment, the location projections 2027 on the frame 2021 may be irregularly spaced, that is, the annular spacing between a pair of adjacent projections is different to the annular spacing between at least one other pair of adjacent projections, such that there is only one angular orientation of the frame in which all of the location projections are able to engage with the respective recesses in the FCPRV frame 2010.

When mounted to the body of the FCPRV 2000, the link connector portion 2025 and/or the diaphragm 2023 of the valve member is aligned with the valve seat 2004 such that in the first configuration of the pressure relief mechanism, the diaphragm and/or the link connector portion seals against the valve seat. Preferably the engagement between the valve seat 2004 and the valve member 2005 is near the periphery of the connector portion 2025, where the connector portion is overmoulded and overlapped by a portion of the diaphragm 2023.

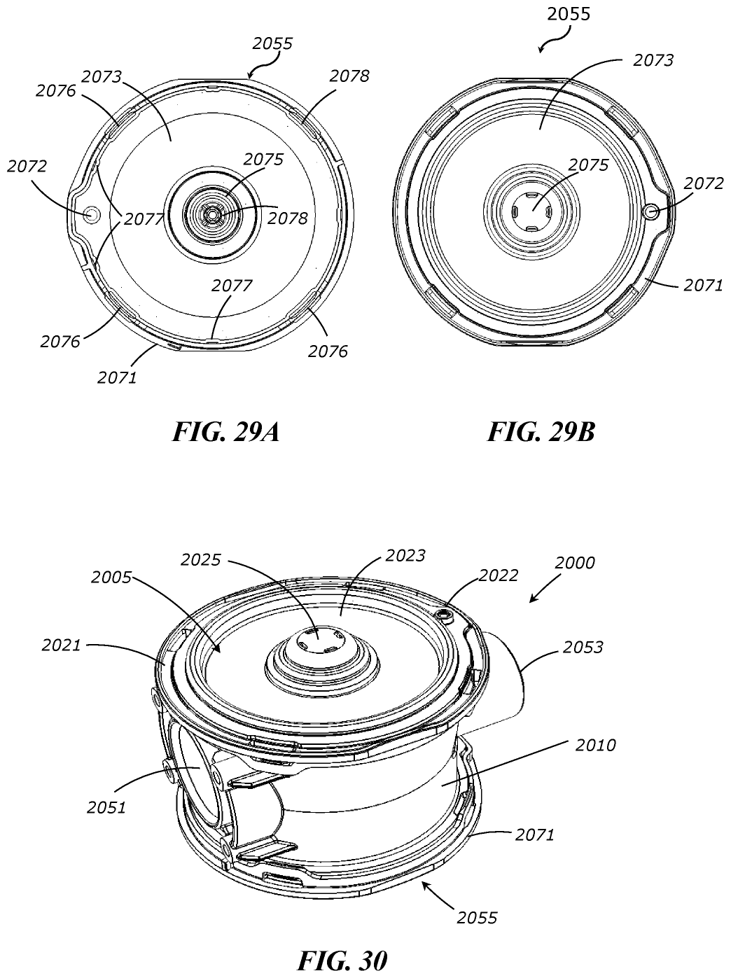

The frame 2021/2071 of each diaphragm component 2005/2055 comprises a port 2022/2072. In the valve member, this port 2022 allows communication of the valve chamber 2002 opposite the valve seat 2004, with atmosphere (the environment exterior to the FCPRV). The port 2022 is sized to receive a rigid cylindrical conduit provided on the body and which defines the passage for communication between the valve chamber 2002 and atmosphere. In the sensing member, the port 2072 provided in the frame 2071 of the diaphragm member 2055 (FIGS. 29A and 29B) allows the second sensing chamber 2054b to communicate with the pressure tap line or communication line 2011, for sensing the flow rate and/or pressure of a gases flow at or through the outlet 2053. The port 2072 is sized to receive the cylindrical conduit that defines the pressure tap line or communication line 2011.

The diaphragm 2023/2073 is a flexible member received into the space defined by the annular frame, with a peripheral portion of the diaphragm over-moulded to the frame. The rigid frame 2021/2071 may be insert moulded with the diaphragm 2023/2073, where a portion of the diaphragm 2023/2073 is over-moulded to the frame 2010. The diaphragm 2023/2073 preferably comprises an elastomeric material, for example thermoplastic elastomer (TPE), LSR (liquid silicone rubber) and compression moulded rubber.

The link connector portion 2025/2075 is a substantially rigid member centrally located relative to the diaphragm frame 2021/2071 and concentric with the annular frame 2021/2071. The connector portion 2025/2075 may be formed from any suitably rigid material, such as a metal or a plastic material, such as polycarbonate or other plastics material known in the art.

The connector portion 2025/2075 is adapted to removably couple to a valve adjustment member such as the mechanical link 2057 described herein. Engagement features are provided on the connector portion 2025 to positively engage with an end portion of the mechanical link 2057.

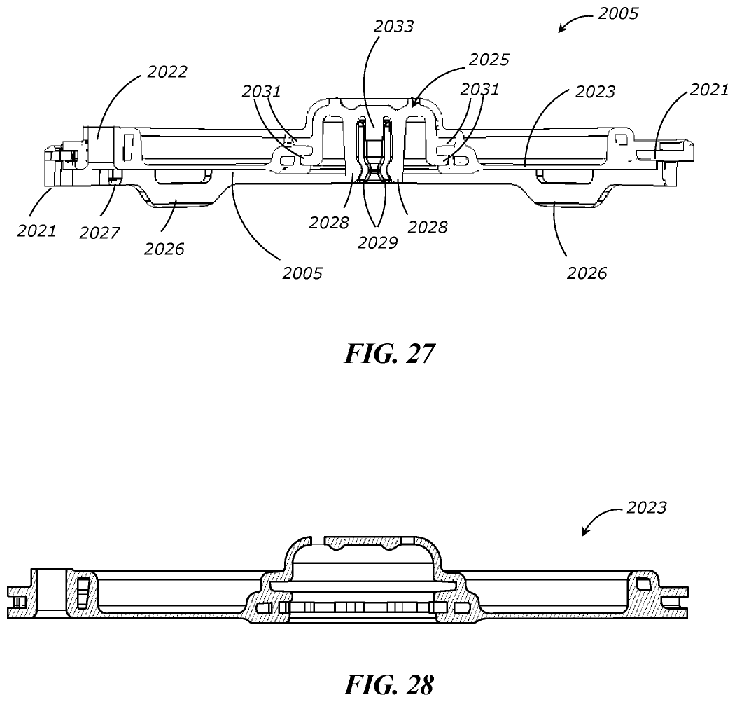

In the embodiment shown, the engagement features comprise four engagement fingers 2028 extending in the first direction from a centre of the connector portion 2025, however other engagement features such as other catches, are possible. The engagement features may comprise more than or fewer than four engagement fingers 2028. Each engagement finger 2027 comprises a protrusion 2029 (FIG. 27) near the free end of the finger, the protrusions protruding in an inward direction, towards a central axis of the diaphragm 2005.

A boss 2033 extends in the first direction a centre of the connector portion 2025, between the engagement fingers. The boss only extends a part of the length of the fingers and is provided to for ease of manufacturing. The boss 2033 supports the end of the mechanical link 2057 while the depth of the boss allows for additional length in the engagement fingers 2028, for greater flexibility of the fingers.

The mechanical link 2057 comprises at least one recess 2030, for example an annular recess proximal to and spaced from each end of the mechanical link. The protrusions 2029 of each connector engagement finger 2028 positively engage the recesses to secure the mechanical link to the connector portion. To join the two components, the end of the mechanical link 2057 is pressed into the space defined by the four connector fingers 2078, with the protrusions contacting a peripheral surface of the mechanical link. The connector engagement fingers 2078 flex as the mechanical link is pressed into position and move back into engagement when the protrusions come into contact with the recess.

In alternative embodiments, the mechanical link may comprise one or more protrusions, for example annular protrusions, and the engagement features on the connector portion may comprise once or more recesses.

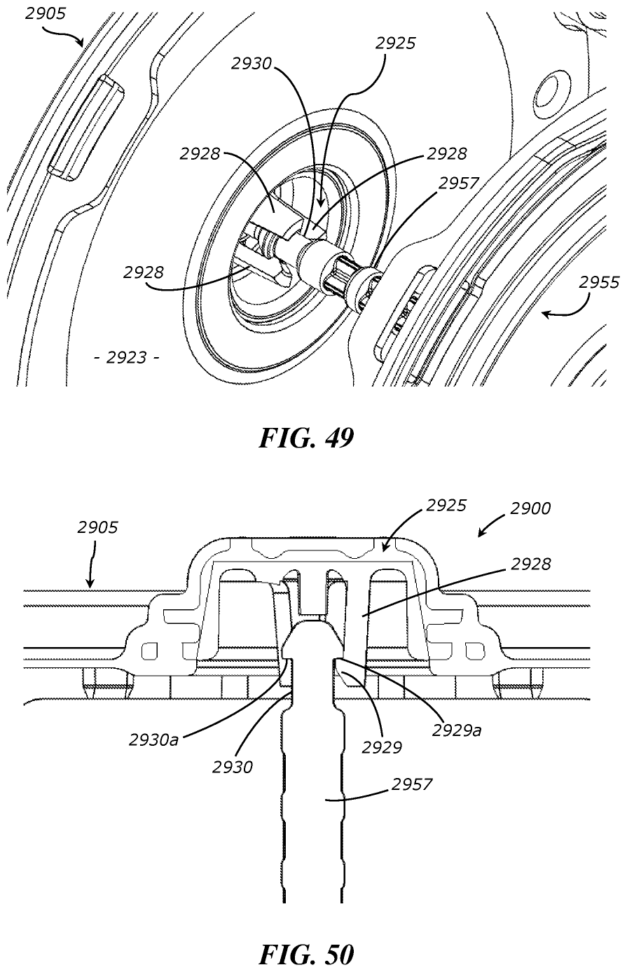

FIGS. 49 and 50 show a valve member 2905 with an alternative embodiment connector portion 2925. Unless otherwise described, the valve member 2905 and mechanical link 2957 have similar features and functionality to the valve member 2005 and link 2057 shown in FIG. 27. Like numbers are used to indicate like parts with the addition of 900.

The connector portion 2925 comprises three engagement fingers 2928 extending in the first direction from a centre of the connector portion 2925, however, alternative embodiments may comprise more or fewer engagement fingers 2928. The inward protrusion 2929 provided near the free end of each engagement finger 2928, is shaped differently to the protrusions 2029 on the engagement fingers in the embodiment of FIG. 27. That is, the protrusions 2929 each comprise a substantially planar surface 2929a which is substantially perpendicular to the longitudinal axis of the mechanical link 2957. The engagement recess 2930 provided at the respective end of the mechanical link 2957 comprises a complementary substantially flat surface 2930a, also substantially perpendicular to the longitudinal axis of the mechanical link 2957, for engaging the flat surface 2929a of the engagement fingers 2928.

The flat surfaces 2929a on the engagement fingers are provided on the portion of the respective protrusion 2929 that is distal to the tip of the engagement finger 2928. The portion of the protrusion 2929 proximal the tip of the engagement finger 2928 comprises a surface that is inclined or curved relative to the mechanical link 2915.

The inclined or curved surfaces of the protrusion 2929 provide a lead-in and allow the fingers 2928 to flex out as the connector portion 2925 is pushed onto the end of the mechanical link 2957 and into engagement with the mechanical link. The perpendicular engagement surfaces 2929a, 2930a then engage and act to resist separation of the mechanical link 2957 from the connector portion 2925. This advantageously prevents inadvertent separation of the mechanical link 2957 from the connector portion during use, particularly when the device is subject to high pressures such as in configurations when the device is not being used to provide pressure relief.

The sensing member 2955 may also comprise a connector portion (not shown) with the engagement features described above, for engaging with the opposite end of the mechanical link 2957.

The link connector portion 2075 of the sensing member can be coupled to the mechanical link 2057 in the same manner as for coupling the valve member 2005 to the mechanical link, but to the opposite end of the mechanical link 2057, thereby coupling the sensing member 2055 and the valve member 2005. When linked, the sensing connector portion 2075, the valve connector portion 2025, and the mechanical link 2057 are substantially co-axial.

The link connector portion 2025/2075 further comprises a pair of spaced apart peripheral flanges 2031. The flanges 2031 are annular and co-axial, and each pair defines an annular space there between to receive the respective diaphragm 2023/2073. The flanges 2031 define an annular space therebetween to receive the respective diaphragm 2023/2073 during the overmoulding process when the diaphragm is overmoulded to the link connector portion 2025/2075.

The link connector portion 2025/2075 is preferably insert moulded with the diaphragm. During the overmoulding process, a portion of the diaphragm fills the annular space defined by the annular flanges 2031 on the connector portion, forming a seal between the link connector portion and the respective diaphragm. This seal advantageously eliminates leakage between the connector portion and the respective diaphragm.

Preferably the diaphragm is over-moulded to both the connector portion and the frame in the same step, to form a single integral diaphragm component 2005/2055. This ensures that the connector portion 2025/2075 is centred relative to the frame and to the diaphragm, thereby ensuring that the mechanical link 2057 is also centred.

In the FCPRV 2000, damping of the valve response is primarily provided through three damping features that provide a resistance to flow. A first damping feature comprises the opening into first sensing chamber 2054a through which a portion of the gases flow from the inlet 2051 to outlet 2053 may enter first sensing chamber 2054a when in use. In the embodiment 2000 of FIG. 24, the opening into chamber 2054a is provided by the tubular guide through which the mechanical link passes. A second damping feature comprises the communication line 2011 that defines a passage between the second sensing chamber 2054b with the main gas flow passage through outlet 2053. The third damping feature comprises the port 2022 and the conduit 2103 to which it engages to define the passage between the valve chamber 2002 and atmosphere. These openings/passages/ports can control flow and the level of damping of the valve response of the FCPRV. Controlling the level of damping can be achieved by altering features of these openings/passages/ports, for example their diameters or shapes. Each of these three openings preferably has a constant diameter so the damping effect is consistent and can be known. Alternatively, the openings may have a tapered or otherwise changing diameter that is known. These openings/passages/ports may comprise a damping feature, e.g. filter to provide additional flow restriction.

In the embodiment of FIG. 24, the mechanical link 2057 comprises a series of transverse ribs and is guided in a tubular guide 2007. The space between the mechanical link and internal wall of the tubular guide is the entry passage for flow from the device inlet 2051 to the first sensing chamber 2054a. This restricted passage and the turbulent flow path created by the ribs creates resistance to flow and has a damping effect on flow onto the sensing mechanism 2050 by reducing the fluctuations in the main gases flow path that reach the sensing member. Damping of the sensing mechanism has a damping effect on movement of the mechanical link, leading to more stable valve operation. However, the amount of damping provided by this arrangement depends on the relative position of the mechanical link 2057 within the tubular guide 2007. If the mechanical link is off centre or not axially aligned with the guide, the damping effect is reduced, and this is not predictable. Rubbing of the mechanical link against the tubular guide also creates hysteresis in the valve, creating a lag in flow being restored in the system after the valve has vented fluid to atmosphere.

The concentric location of the connector portion 2025/2075 created by the integral, over-moulded diaphragm components helps to consistently hold the mechanical link 2057 centrally within the tubular guide 2007, for improved, more predictable damping and reduced hysteresis.

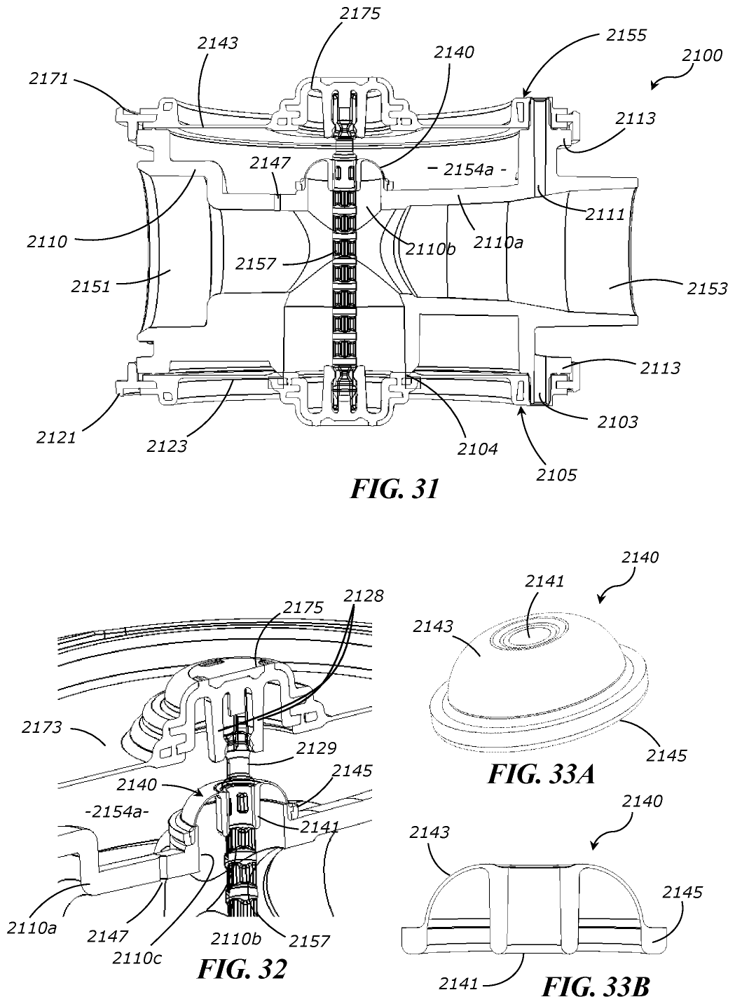

FIG. 31 shows a further embodiment FCPRV 2100. The FCPRV 2100 has similar features and functionality to the FCPRV 2000 of FIG. 24, unless described below. Like numbers are used to indicate like parts with the addition of 100.

The FCPRV 2100 comprises a device inlet 2151 and a device outlet 2153, with a main gas flow passage between the inlet and the outlet. A pressure relief mechanism between the inlet and the outlet comprises valve seat 2104 and a valve member, which operates substantially as described above in relation to the previous embodiment 2000 to vent at least a portion of a flow of gases through the gas flow passage when the flow of gases exceed a pressure threshold. The valve member comprises a diaphragm component 2105 as described above, but other embodiments may comprise alternative valve arrangements.

A sensing mechanism dynamically adjusts the pressure threshold based on a flow rate and/or pressure of a portion of the flow of gases through the gas flow passage. The sensing mechanism comprises a sensing member in the form of a diaphragm component 2155, as described above in relation to the previous embodiment, but other embodiments may comprise alternative sensing member arrangements.

The sensing mechanism comprises a mechanical link 2157 coupling the pressure relief valve and the sensing diaphragm component 2155, and a sealing boot 2140 to substantially seal against the mechanical link 2157.

The sealing boot 2140 is a flexible component, for example comprising an elastomeric material, and is illustrated in more detail in FIGS. 32 to 33B. The sealing boot 2140 is attached to the mechanical link 2157, at a point on the link proximal to and spaced from the connection to the sensing diaphragm component 2155. The sealing boot 2140 defines a central channel 2141 for receiving the mechanical link 2157. The walls of the channel 2141 seal against the mechanical link to substantially prevent or reduce fluid flow between the mechanical link 2157 and the sealing boot 2140.

The diameter of the opening defined by the sealing boot 2140, when the sealing boot 2140 is not installed, may be slightly less than the outer diameter of the mechanical link 2157 such that inserting the mechanical link into the channel 2141 of the sealing boot causes the channel to expand into a tensioned state thereby enhancing the connection and seal between the sealing boot and the mechanical link. The outer surface of the mechanical link at least at the connection between the mechanical link 2157 may be substantially cylindrical and smooth to enhance the connection between the mechanical link 2157 and the sealing boot 2140. In the embodiment shown, a major part of the length of the mechanical link 2157 comprises a ribbed surface, however, in alternative embodiments the mechanical link may not have any ribs and may instead be substantially smooth.

A first sensing chamber 2154a adjacent to the sensing diaphragm 2173 is defined by a wall 2110a of the valve body 2110. The wall defines a first aperture 2110b for receiving the mechanical link 2157, the first aperture 2110b being wider than the mechanical link. The sealing boot 2140 extends across the aperture 2110 and seals against the wall 2110a.

In the embodiment shown, the rim of the first aperture 2110b comprises a lip or flange extending substantially perpendicular to the sensing chamber wall 2110a. This lip 2110c acts as a retention mechanism to retain the sealing boot 2140 to the aperture 2110b. A base 2145 of the sealing boot 2140 extends around the lip, abutting against the lip to for a seal. The inner diameter of the base 2145 of the sealing boot 2140, when the sealing boot 2140 is not installed, may be slightly less than the outer diameter of the lip 2110c of the aperture rim, such that inserting the sealing boot 2140 over the lip causes the base of the sealing boot to expand into a tensioned state thereby enhancing the connection between the sealing boot and the lip 2110c. The base 2145 of the sealing boot 2140 comprises a thickened lip region to provide a tighter seal with the aperture lip.

The damping diaphragm 2140 comprises a flexible body 2143 extending from the base 2145 of the sealing boot to the centre channel 2141. In the embodiment shown, the flexible body is curved, in a convex manner relative to the first chamber. This curved flexible body 2145 allows axial movement of the mechanical link 2157 relative to the wall 2110 of the sensing chamber 2154a, while maintaining the seal between the mechanical link and the wall. The mechanical link 2157 can move through a movement range to provide a desired range of adjustment of the bias of the valve member.

The sealing boot 2140 provides negligible resistance to axial movement through the movement range. That is, the mechanical link can move axially through the desired movement range, substantially unimpeded by the sealing boot 2140.

The sealing boot 2140 is resilient and resistant to buckling under the axial loads sufficient to adjust the valve mechanism. In alternative embodiments, the sealing boot 2140 may comprise a concertina membrane rather than a curved wall to allow axial movement of the mechanical link.

The sealing boot 2140 may be formed from any suitably flexible material, such as an elastomeric or a plastic material, for example thermoplastic elastomer (TPE), LSI (liquid silicone rubber), compression molded rubber, or another suitable material known in the art. Alternatively, one or more portions of the sealing boot 2140 may comprise a substantially rigid material such as polypropylene, and a living hinge, to enable flexing of the boot.

In the embodiment shown 2100, no guide channel is provided for the mechanical link 2157. A guide channel between the sensing diaphragm and the valve diaphragm is not necessary because flow along the mechanical link is substantially sealed off between the gas flow passage and the first sensing chamber 2154a, the guide channel is not required for damping purposes. However, in alternative embodiments a guide channel may be provided for the mechanical link with the mechanical link being axially slidable in the channel and the sealing boot arranged at the end of the guide channel nearest the sensing diaphragm. In alternative embodiments, the sealing boot may be provided along the guide channel, for example a portion of the sealing boot may engage with a wall of the guide channel and another portion of the sealing boot may seal against the mechanical link.

The first sensing chamber 2154a adjacent to the sensing diaphragm is in fluid communication with the inlet 2151 to sense the pressure upstream of the flow restriction. Since the space around the mechanical link 2157 is sealed by the sealing boot 2140, a passage into the first sensing chamber 2154a is provided elsewhere. In the embodiment shown, a damping aperture 2147 is provided in the chamber wall 2110a to allow fluid communication with the inlet 2151.

Preferably the passage 2147 into the first sensing chamber 2154a from the inlet 2151 is small and/or restricted so create a resistance to flow and damp flow into the sensing mechanism 2050 by reducing the fluctuations from the main gas flow passage that reach the sensing member. Damping of the sensing mechanism has a damping effect on movement of the mechanical link, leading to more stable valve operation. In the embodiment shown, the damping aperture 2147 has a diameter of between 0 mm and 10 mm, with a smaller aperture providing increased damping. In alternative embodiment, the wall 2154a of the sensing chamber may comprise a plurality of damping apertures. In embodiments having a plurality of damping apertures, the apertures may be smaller than in embodiments with a single damping aperture, to provide a similar level of damping. The wall 2154a may further comprise a boss at each aperture through which each aperture extends, extending the length of the channel defined by the aperture and thereby increasing the resistance to flow through the aperture.

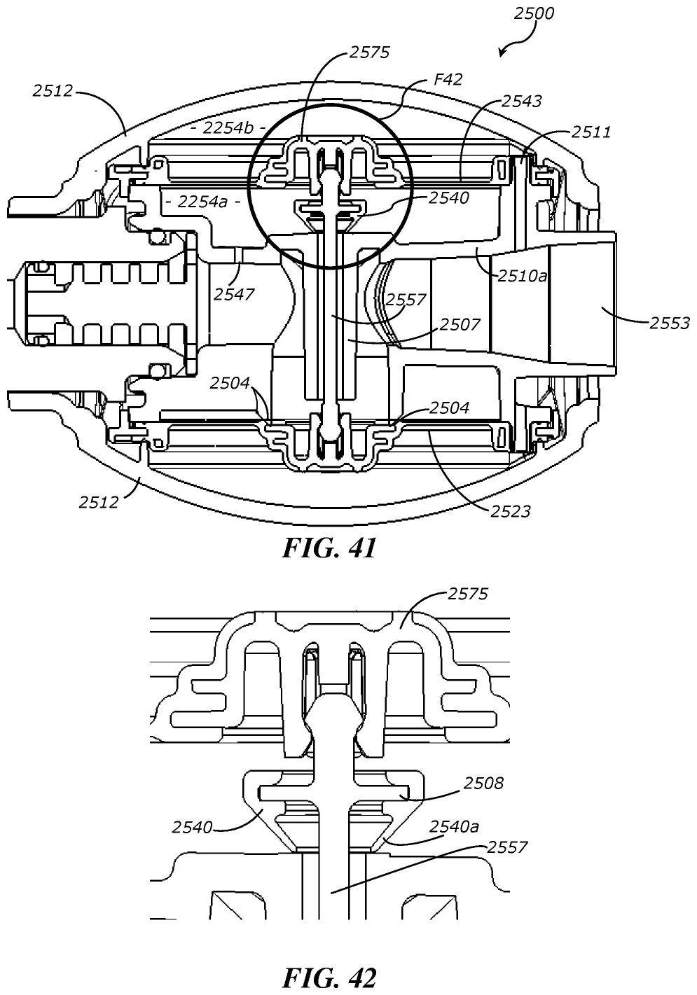

FIG. 41 shows a further embodiment FCPRV 2500. The FCPRV 2500 has similar features and functionality to the FCPRV 2100 of FIG. 31, unless described below. Like numbers are used to indicate like parts with the addition of 400.

In this embodiment, a guide channel 2507 may optionally be provided for the mechanical link 2557, with a clearance provided between the surface of the mechanical link and the inner surface of the guide channel. Preferably the clearance is more than 0 mm, and more preferably the clearance is about 1 mm. A sealing boot 2540 is provided on the mechanical link 2557 to prevent gasses flowing out of the guide channel 2507, into the first sensing chamber 2554a.

Referring to the detail view of FIG. 42, the boot 2540 is mounted to the mechanical link 2557 such that the boot 2540 moves in tandem with the mechanical link. The boot 2540 is mounted to the mechanical link via an outwardly extending annular flange 2508 on the mechanical link. The boot 2540 has a complementary annular recess on an inner surface that receives the flange. The boot 2540 is a flexible, resilient member, preferably comprising an elastomer. Use of an elastomer advantageously enables the boot 2540 to be stretched over the flange 2508 to assemble the boot and mechanical link. Compression forces in the boot 2540 keep the boot engaged with the flange 2508 to substantially seal the connection between the boot and the mechanical link.

The boot 2540 comprises a tapered portion 2540a with an edge that abuts a surface of the valve body wall 2510 that defines the first sensing chamber 2554a. The tapered portion 2540a abuts the chamber wall 2510a around the end opening of the guide channel 2507.

The valve seat 2504 opposite the sealing boot 2540, prevents the mechanical link 2557 moving towards the sensing member 2543, away from the valve seat 2504 and thereby prevents the boot 2540 from lifting out of contact with the chamber wall 2510a. The inward taper of the tapered portion 2540a and the resilient nature of the boot 2540 ensures the boot 2540 remains in contact with the chamber wall 2510a to substantially seal flow into the sensing chamber 2554a from the guide channel 2507 when the valve 2523 lifts from the valve seat 2504 and when it lowers again. The wall of the tapered portion 2540 of the boot 2540 is thinner than the wall thickness of the portion of the boot adjacent the flange 2508. This reduced thickness minimises any resistance to axial movement from the boot 2540.

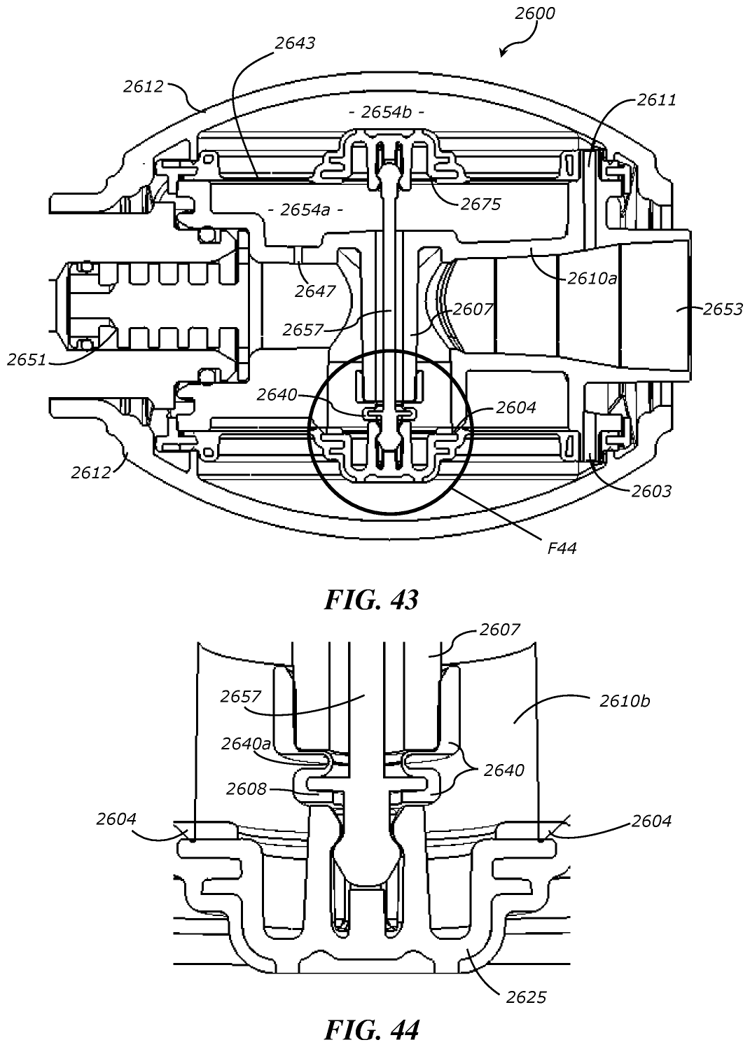

FIG. 43 shows a further embodiment FCPRV 2600. The FCPRV 2600 has similar features and functionality to the FCPRV 2100 of FIG. 31, unless described below. Like numbers are used to indicate like parts with the addition of 500.

In this embodiment, the sealing boot 2640 seals off is provided at the end of the mechanical link guide channel 2607 proximal to the valve diaphragm 2623, thereby preventing gas flow into the guide channel from the main passage. The guide channel 2607 is instead in fluid communication with the first sensing chamber 2654a.

Referring to the detail view of FIG. 44, a first portion of the sealing boot 2640 is mounted to the mechanical link 2657 to move in tandem with the mechanical link, and a second portion of the sealing boot 2640 is mounted to the guide channel 2607.

The boot 2640 is mounted to the mechanical link via an outwardly extending annular flange 2608 provided on the mechanical link 2657. In alternative embodiments, the boot 2640 could be attached to the mechanical link in other ways, for example, by over-moulding the boot to the link. The boot 2540 has a complementary annular recess on an inner surface that receives the flange 2608. The boot 2640 is a flexible, resilient member, preferably comprising an elastomer. Use of an elastomer advantageously enables the boot 2640 to be stretched over the flange 2608 to assemble the boot and mechanical link 2657, and over the guide channel 2607 to assemble the boot 2640 to the guide channel 2607. Compression forces in the boot 2640 keep the boot 2640 engaged with the flange 2508 and the guide channel 2607 to substantially seal the respective connections. In alternative embodiment, the guide channel may be shorter than the one in the embodiment shown, and the boot 2640 could be positioned nearer the sensing member 2643.

The boot 2640 comprises a necked portion 2640a between the two connection portions with a wall thickness that is thinner than the wall thickness of the portion of the boot adjacent the flange 2508. The necked portion 2640 comprises is U-shaped in cross section or is otherwise concertinaed to allow the mechanical link and guide channel connection portions to move away from each other. As the connection portions move away from each other, the necked portion 2640a straightens and as the connection portions move back towards each other, the bend in the necked portion again increases. During normal use, this prevents the transfer of tension between the mechanical link and the guide channel.

Both the embodiments 2500, 2600 of FIGS. 41 to 44, one or more damping apertures 2547, 2647 is provided in the wall 2510a, 2610a of the first sensing chamber. In these embodiments, the damping arrangement is provided by the combination of the damping aperture and the sealing boot.

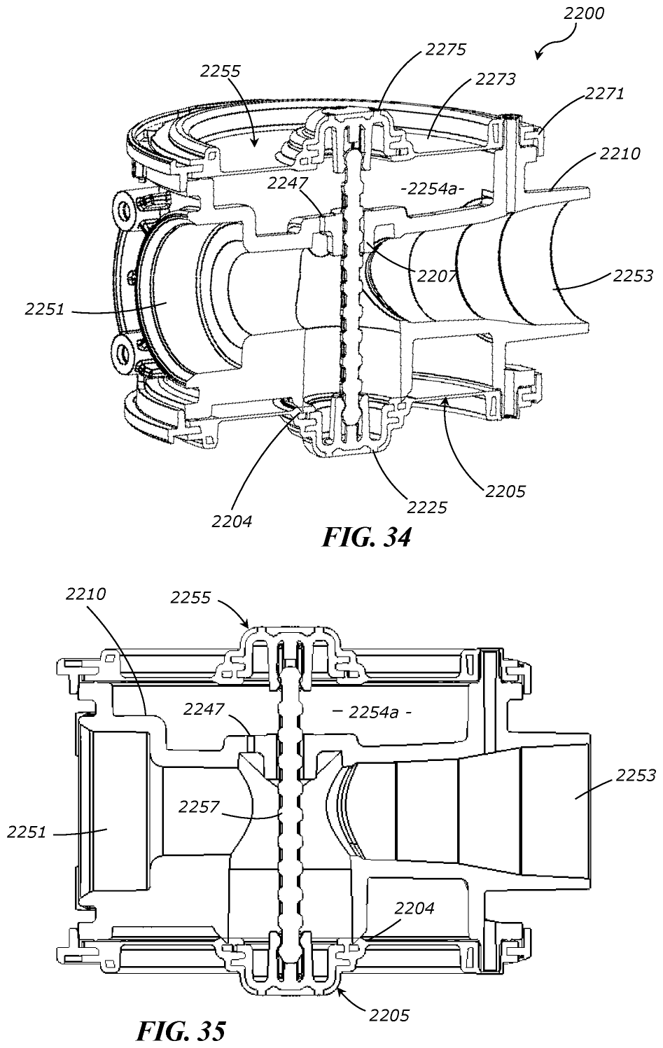

As an alternative to a sealing boot, other embodiment FCPRVs may comprise alternative means to seal around the mechanical link to prevent fluid leaking into the first sensing chamber along the mechanical link. FIGS. 34 and 35 illustrate one embodiment FCPRV 2200, in which the mechanical link is guided within a guide channel 2207. The FCPRV 2200 has similar features and functionality to the FCPRV 2100 of FIGS. 31 and 32, unless described below. Like numbers are used to indicate like parts with the addition of 100.

The guide channel 2207 is a tubular guide. In the embodiment shown, the length of the tubular guide is short in comparison to the length of the mechanical link, for example, less than about 25% of the length of the link. However, in alternative embodiments, the guide may be longer, for example the guide may extend along a majority of the length of the link.

To seal around the mechanical link 2257 to prevent gases leaking into the first sensing chamber 2254a along the mechanical link, the guide channel 2207 contains a viscous fluid, for example grease. The viscous fluid fills the space between the internal surface of the guide channel and the surface of the mechanical link, and allows the mechanical link 2257 to move axially within the channel 2207 while sealing the channel to prevent gas flow along the guide channel. The viscous fluid is preferably a low shear fluid to minimise any resistance to axial movement of the mechanical link. However, in some embodiments, the viscous fluid may additionally damp movement of the mechanical link by providing resistance to axial movement of the link.

Preferably the fluid is a low strength, high viscosity fluid that shears readily but which demonstrates a high resistance to shear forces. In some embodiments, the fluid is one having Bingham plastic characteristics, for example with fixed shear strength; or a dilatant fluid with non-Newtonian properties, for example in which the viscosity increases with the applied shear stress.

The viscous fluid provides a predictable amount of hysteresis to the FCPRV. A higher level of damping is generally provided through use of higher viscosity fluid. If reduced static forces and residual tension is desired, a thinner layer of viscous fluid (for, example using a narrower guide channel), or a shorter section of grease (for example, through use of the shorter guide channel 2207) can be used.

In some embodiments, a membrane or a seal may optionally be provided at one or both ends of the channel to contain the viscous fluid within the channel, while still allowing axial movement of the mechanical link.

A damping aperture 2247 is provided in the chamber wall 2110a to allow fluid flow into the first sensing chamber 2254a from the valve inlet 2251. The damping aperture may comprise a filter such as a porous material over the aperture to create increased resistance to flow through the aperture 2247.

Preferably the passage 2247 into the first sensing chamber 2254a from the inlet 2251 is small and/or restricted so create a resistance to flow and damp flow into the sensing mechanism 2250 by reducing the fluctuations from the main gases flow path that reach the sensing member. Damping of the sensing mechanism has a damping effect on movement of the mechanical link, leading to more stable valve operation. In alternative embodiment, the wall 2154a of the sensing chamber may comprise a plurality of damping apertures.

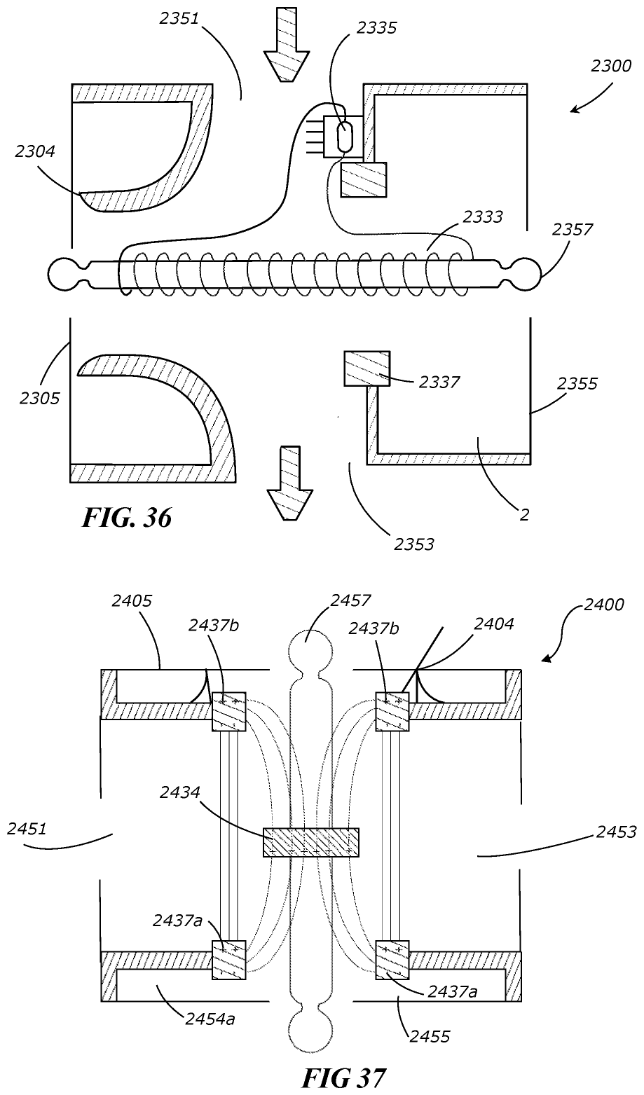

FIGS. 36 and 37 schematically illustrate two embodiment FCPRVs 2300, 2400, comprising magnetic arrangements to damp movement of the mechanical link 2357, 2457. The FCPRVs 2300, 2400 have similar features and functionality to the FCPRV 2100 of FIGS. 31 and 32, unless described below. Like numbers are used to indicate like parts with the addition of 200 or 300, respectively.

In a first embodiment shown in FIG. 36, the magnetic arrangement comprises a conductive coil 2333 extending along a length of the mechanical link 2357, which couples the sensing diaphragm 2355 to the valve diaphragm 2305. The conductive coil is electrically connected to an electrical resistor.

A magnet is arranged to induce an electrical current in the coil upon axial movement of the mechanical link. The magnet is in the form of a ring and surrounds the mechanical link. The magnet may be a permanent magnet or an electromagnet.

The electrical resistor dissipates heat generated by the induced current in the coil. The electrical resistor provides a ‘load’ for the induced current which creates an effective resistance against the movement of the mechanical link and conductive coil, thereby damping movement of the pressure relief valve and/or the sensing mechanism. In an alternative embodiment shown in FIG. 37, the magnetic arrangement comprises an electrically conductive member mounted to the mechanical link 2457. In this embodiment, the electrically conductive member is a ring 2434 comprising an electrically conductive material such as copper. The ring 2434 is fixed to the mechanical link 2457 at a point intermediate the opposing ends of the mechanical link, for example at a mid-point of the link.

First and second magnets 2437a, 2437b, are provided within the body of the pressure relief device 2400, and fixed relative the body. In the embodiment shown, the first and second magnets 2437a, 2437b are ring magnets, encircling the mechanical link 2457 such that the mechanical link 2457 is axially movable within the ring openings.

Each ring magnet 2437a, 2437b defines a positive pole and a negative pole. The ring magnets are positioned such that the positive pole of the first ring magnet 2437a is nearest the negative pole of the second ring magnet 2437b, to thereby create a magnetic field extending between the first and second ring magnets.

The ring magnets 2437a, 2437b are preferably the same size and strength and are arranged to be coaxial and spaced apart. The conductive ring 2434 on the mechanical link 2457 is positioned intermediate the two ring magnets 2437a, 2437b, in the created magnetic field. The magnetic field provides resistance to movement of the conductive ring 2434 towards one of the ring magnets 2437a, 2437b, thereby providing resistance to movement of the mechanical link 2457.

The first and second ring magnets 2437a, 2437b may comprise electromagnets, with the strength of the magnetic field being adjustable by altering a current through the electromagnets. Alternatively, the ring magnets 2437a, 2437b may be permanent magnets.

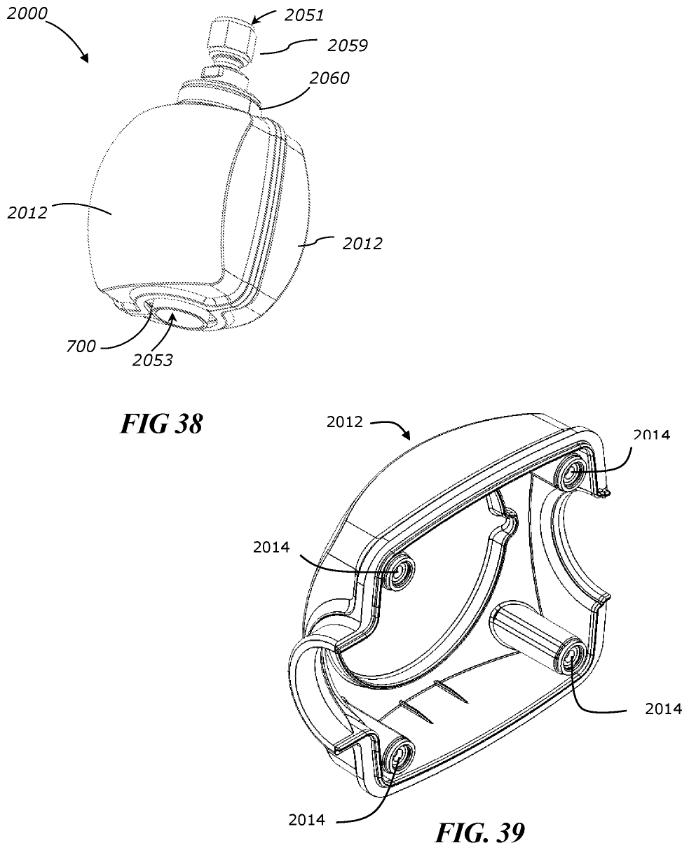

FIG. 38 is a view of a pressure relief valve described above, showing the valve housing which comprises two chamber caps 2012. The valve chamber caps 2012 are secured in place to cover the valve body and the inner components of the valve and form the second valve and sensing chambers. The valve housing comprises ribs or other location features on an internal surface of each cap 2012 to assist with correctly locating the valve body 2010 within the caps 2012. These ribs or location features assist with accurately positioning the valve body within the housing. Accurate positioning is important because a first one of the chamber caps 2012 defines a wall of the second sensing chamber 2154b that is required for flow and/or pressure compensation pressure relief in the valve. Misalignment of the valve body and the chamber caps could cause variations in the sensing chamber that could result in inconsistent or unreliable flow and/or pressure compensation.

In some embodiments the two chamber caps 2012 may be ultrasonically welded together to prevent access to the interior of the FCPRV. Preventing access to the valve can help to ensure that the function of the valve, including the flow and/or pressure compensation, is not deliberately or inadvertently altered, for example through servicing of the valve.

In other embodiments the two housing caps 2012 may be ultrasonically welded, screwed or otherwise permanently or removably fastened together. In the embodiment shown in FIG. 39, the chamber caps 2112 each provide a plurality of apertures 2014 for the receipt of fasteners such as threaded fasteners. Where removable fasteners are used, the heads of the fasteners may be covered, for example using screw caps or plugs, to disguise/hide the screws and deter general access to the interior of the FCPRV.

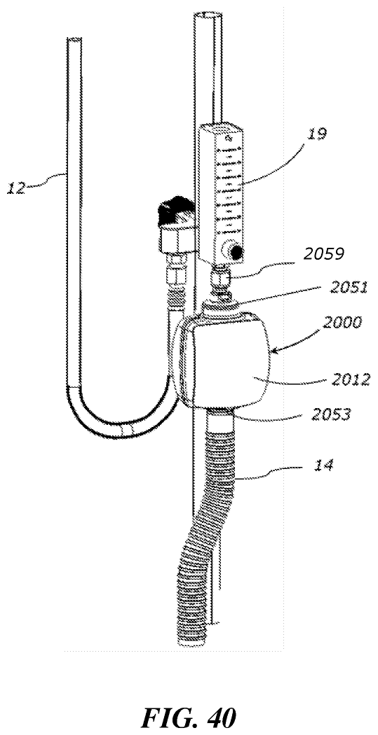

As illustrated in FIG. 40, in use, the pressure relief valve is preferably arranged in a vertical orientation during use or operation, where the longitudinal axis of the valve which extends from the inlet 2151 to the outlet 2153, is substantially perpendicular to a ground surface. In a vertical orientation, the sensing and valve diaphragms lie in substantially vertical planes. This eliminates or substantially reduces the impact of gravitational forces on the operation of the diaphragms. Gravity may otherwise impact the valve in a horizontal orientation due to the self-weight of the diaphragm components and the self-weight of the mechanical link. In other embodiments, the pressure relief valve is arranged in a horizontal orientation during use or operation, where the longitudinal axis of the valve is substantially parallel to a ground surface. In some embodiments, the pressure relief valve is arranged in an inclined orientation where the longitudinal axis of the valve is at an angle to a ground surface. When the valve is in a vertical or inclined orientation, the inlet 2153 is preferably arranged above the outlet 2153. In other embodiments, the outlet 2153 is arranged above the inlet 2151 when the valve is in a vertical or inclined orientation. Advantageously, a vertical orientation prevents any liquids that may be present in the system from entering the valve relief outlet and potentially impacting gas flow through the valve. The vertical orientation of the valve also allows the flange 2060 of the coupler 2059 to be positioned over the inlet 2151, providing a surface for liquids to fall off without entering the interior of the valve via the inlet 2151.

The inlet 2051 is preferably positioned above the outlet 2053, and coupled to a gas supply 12 via a flow meter 19. The gas supply 12 may be a wall gas source. The outlet 2053 is positioned below the inlet 2051 and is coupled to a conduit 14 for the supply of gases exiting the outlet 2053 to a patient. In the arrangement shown, the inlet 2051 and outlet 2053 are coaxial and vertically aligned.

1. A connector comprising:
a connector body having an inlet and an outlet defining a gas flow passage therebetween;
the connector body having an overlap portion that is overlapped by a portion of a second connector when connected; and
an access passage extending through the overlap portion to the gas flow passage.

2. The connector according to claim 1, wherein the access passage comprises an aperture that fluidly communicates with the gas flow passage to sense pressure in the gas flow passage.

3. The connector according to claim 2, wherein the gas flow passage is defined at least in part by a wall of the connector, and the aperture is in the wall of the connector.

4. The connector according to claim 1, further comprising a cavity forming portion that forms a cavity with the second connector when connected.

5. The connector according to claim 4, wherein the cavity forming portion comprises an arcuate surface.

6. The connector according to claim 4, wherein the cavity forming portion is a recess in a surface of the connector body.

7. The connector according to claim 4, wherein the cavity forming portion is in fluid communication with the gas flow passage via the access passage.

8. The connector according to claim 4, wherein the cavity forming portion has a longitudinal dimension that is substantially parallel to a direction of gas flow in the gas flow passage.

9. The connector according to claim 4, further comprising a first sealing mechanism that forms a first seal with a first portion of the second connector when connected.

10. (canceled)

11. (canceled)

12. The connector according to claim 9, wherein the first sealing mechanism comprises an internal or external sealing surface for friction/interference fit with the second connector.

13. The connector according to claim 9, wherein at least one of the access passage and the cavity forming portion is arranged upstream of the first sealing mechanism.

14. The connector according to claim 9, further comprising a second sealing mechanism that forms a second seal with a second portion of the second connector when connected.

15. The connector according to claim 14, wherein at least one of the cavity forming portion and the access passage is between the first sealing mechanism and the second sealing mechanism.

16. (canceled)

17. (canceled)

18. (canceled)

19. The connector according to claim 14, wherein the second sealing mechanism comprises an internal or external sealing surface for friction/interference fit with the second connector.

20. The connector according to claim 1, wherein a portion of the connector is tapered.

21. (canceled)

22. The connector according to claim 2, wherein the aperture is arranged substantially parallel or substantially perpendicular to a direction of gas flow in the gas flow passage.

23. The connector according to claim 2, wherein the aperture is radially arranged about the gas flow passage.

24. The connector according to claim 2, wherein the connector further comprises a stepped portion and the aperture is arranged on the stepped portion.

25. (canceled)

26. The connector according to claim 1, further comprising a flow restriction.

27. (canceled)

28. (canceled)

29. (canceled)

30. (canceled)

31. (canceled)

32. The connector according to claim 1, further comprising a stop.

33-115. (canceled)

116. The connector according to claim 1, wherein the inlet receives gas flow from a flow source and wherein the gas flow travels through the gas flow passage from the inlet to the outlet.

117. The connector according to claim 1, further comprising an inlet end and an outlet end opposite the inlet end, wherein the inlet end is smaller than the outlet end.

118. An assembly comprising:
a first connector comprising a connector body having an inlet, an outlet, a gas flow passage between the inlet and the outlet, and an overlap portion;
a second connector comprising a connector body having an inlet, an outlet, a gas flow passage between the inlet and the outlet of the connector body of the second connector, and an overlap portion, wherein the first and second connectors are connectable to one another such that the overlap portion of the first connector is received by the overlap portion of the second connector thereby forming an assembly gas flow passage;
an access passage extending through the overlap portion of the connector body of the first connector to the assembly gas flow passage.
